(19) United States
(12) Patent Application Publication
(10) Pub. No.: US 2024/0167031 A1
(43) Pub. Date: May 23, 2024

(54) NUCLEIC ACID DELIVERY TO THE CENTRAL NERVOUS SYSTEM

(71) Applicant: Biogen MA Inc., Cambridge, MA (US)

(72) Inventors: Hasan M. Yildiz, Holly Springs, NC (US); Mandana Bornapour, Arlington, MA (US); Paul Peng, Belmont, MA (US); Vishwesh A. Patil, Arlington, MA (US); Brian R. Simler, Westford, MA (US); William F. Kiesman, Wayland, MA (US)

(21) Appl. No.: 18/283,925

(22) PCT Filed: Mar. 31, 2022
(86) PCT No.: PCT/US2022/022751

§ 371 Date: Sep. 25, 2023

Related U.S. Application Data
(60) Provisional application No. 63/169,539, filed on Apr. 1, 2021.

(51) Int. Cl. C12N 15/113; A61K 9/00; A61K 9/51; A61K 47/59

(52) U.S. Cl. CPC C12N 15/113; A61K 9/0019; A61K 9/0085; A61K 9/5153; A61K 47/59; C12N 2310/11; C12N 2310/315; C12N 2310/322; C12N 2310/3341; C12N 2310/335; C12N 2310/345

(57) ABSTRACT

Featured are polymeric nanocarriers (e.g., PLGA nanoparticles) with encapsulated nucleic acid (e.g., an antisense oligonucleotide) for delivery (e.g., intrathecally) to the central nervous system. These polymeric nanocarriers are useful in the treatment of central nervous system disorders. They are capable of delivering their cargo (e.g., an antisense oligonucleotide) in higher amounts, for a longer period of time, and into deeper regions of the brain than a free or unformulated antisense oligonucleotide. The efficient delivery and distribution of antisense oligonucleotides results in reducing the number of administrations and patient compliance and improves patient experience.

### CROSS-REFERENCE TO RELATED APPLICATIONS

This application claims the right of priority to U.S. Provisional Appl. No. 63/169,539 filed Apr. 1, 2021, the contents of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on Mar. 25, 2022, is named 13751-0301WO1_SL.txt and is 10,722 bytes in size.

### TECHNICAL FIELD

This disclosure relates generally to compositions for delivering a therapeutic agent to the central nervous system and methods of using same for treatment of neurological disorders.

### BACKGROUND

The delivery of drugs to the central nervous system (CNS) has been a challenge in the treatment of neurological diseases such as Alzheimer's disease and Parkinson's disease. For drugs to reach the nervous system they first have to penetrate the blood brain barrier (BBB), which is a major challenge due to the selectivity of the BBB. The BBB acts as a semipermeable membrane, preventing most molecules from entering the nervous system from the blood and allows only low molecular weight (<400 Da) and lipophilic compounds to pass. Most small molecules and large molecules, such as monoclonal antibodies and antisense oligonucleotides, cannot pass through this barrier. Due to this challenging process of drug penetration across the BBB, less than 10% of therapeutic agents for neurological diseases make it to clinical trials.

One way to access the CNS directly is through the use of intrathecal (IT) injection. By injecting directly into the cerebrospinal fluid (CSF), a therapeutic has direct access to the CNS. However, the CSF is turned over several times a day, so the residence time of the therapeutic can be limited. There is a need in the art for improved methods for delivering a therapeutic agent to the CNS.

### SUMMARY

This application relates in part to compositions for delivering a therapeutic agent (e.g., a nucleic acid such as an antisense oligonucleotide) to the central nervous system (CNS). Also featured are methods of treating neurological diseases using such compositions. In addition, provided are methods of increasing the amount and/or residence time of a therapeutic agent delivered to the brain of a human subject. Furthermore, this disclosure relates to methods of delivering a therapeutic agent deeper into the brain of a human subject in need thereof.

In one aspect, the disclosure features a central nervous system (CNS) delivery composition. The composition includes a polymeric nanocarrier and an antisense oligonucleotide. The antisense oligonucleotide is encapsulated within the polymeric nanocarrier and is directly pre-complexed with a counter agent—a cationic molecule-prior to encapsulation.

In some instances, the polymeric nanocarrier is selected from the group consisting of poly(l-lactide), poly(glycolide), poly(d, l-lactide) (PLA), poly(dioxanone), poly(d, l-lactide-co-l-lactide), poly(d, l-lactide-co-glycolide), poly(glycolide-co-trimethylene carbonate), poly(caprolactone) (“polycaprolactone”), poly(d, l-lactide-co-glycolide) (PLGA), poly(dioxanone) poly(glycolide-co-trimethylene carbonate), and mixtures thereof. In one instance, the polymeric nanocarrier is PLGA. In instances, where the CNS delivery composition is a PLGA nanoparticle, the PLGA nanoparticle comprises lactic acid:glycolic acid in a ratio in the range of 2:98 to 98:2. In certain instances, the PLGA nanoparticles comprise lactic acid:glycolic acid in a ratio selected from the group consisting of: 2:98, 3:97, 4:96, 5:95, 6:94, 7:93, 8:92, 9:91, 10:90, 11:89, 12:88, 13:87, 14:86, 15:85, 16:84, 17:83, 18:82, 19:81, 20:80, 21:79, 22:78, 23:77, 24:76, 25:75, 26:74, 27:73, 28:72, 29:71, 30:70, 31:69, 32:68, 33:67, 34:66, 35:65, 36:64, 37:63, 38:62, 39:61, 40:60, 41:59, 42:58, 43:57, 44:56, 45:55, 46:54, 47:53, 48:52, 49:51, 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:4, 98:2, and 100:0.

The counter agent is a cationic molecule that forms a complex with the antisense oligonucleotide. In some instances, the cationic molecule is a cationic peptide. In other instances, the cationic molecule is chitosan. In some instances, the cationic molecule is hexadecylamine. In some instances, the cationic molecule is lauric arginate. In yet other instances, the cationic molecule is a polyethylene imine (PEI). In some cases, the PEI is a linear PEI. In other cases, the PEI is a cross-linked PEI.

In some instances, the CNS delivery composition further includes a therapeutic agent. In certain cases, the therapeutic agent is selected from the group consisting of a small molecule, a cDNA, an mRNA, an siRNA, an miRNA, an aptamer, a ribozyme, and a different antisense oligonucleotide.

In certain instances, the CNS delivery composition is formulated for intrathecal delivery to a human subject.

In some instances, the antisense oligonucleotide is a gapmer or a splice switching antisense oligonucleotide. In some instances, the antisense oligonucleotide is one that is useful in the treatment of a neurodegenerative disease (e.g., a tauopathy, a synucleinopathy). In certain cases, the antisense oligonucleotide comprises or consists of a nucleic sequence set forth in SEQ ID NO:1. In one instance, the antisense oligonucleotide consists of 18 linked nucleosides, wherein the oligonucleotide has a nucleobase sequence consisting of the nucleobase sequence ^{Me}U^{Me}CA^{Me}C^{Me}U^{Me}U^{Me}U^{Me}CA^{Me}UAA^{Me}UG^{Me}C^{Me}UGG (SEQ ID NO:1), wherein each internucleoside linkage of the oligonucleotide is a phosphorothioate linkage, each nucleoside of the oligonucleotide is a 2′-methoxyethyl nucleoside, ^{Me}U is a 5-methyl-uracil, and ^{Me}C is a 5-methylcytosine.

In another aspect, the disclosure relates to a method of treating a CNS disorder in a human subject in need thereof. The method involves administering to the human subject a therapeutically effective amount of a CNS delivery composition described above.

In some instances, the administering is by intrathecal injection. In certain cases, the intrathecal injection is a bolus injection. In some cases, the CNS disorder is a synucleinopathy or a tauopathy. In certain instances, the CNS disorder is spinal muscular atrophy (SMA), amyotrophic lateral sclerosis (ALS), Parkinson's disease, Alzheimer's disease, Huntington's disease, Angelman syndrome, frontotemporal dementia (FTD), Creutzfeldt-Jakob disease, spinocerebellar ataxia type 3 (SCA3), or Menkes disease.

In another aspect, the disclosure features a method of treating SMA, increasing inclusion of exon 7 in SMN2 messenger ribonucleic acid (mRNA) transcripts in a human subject having loss of both functional copies of the SMN1 gene, or increasing exon 7 inclusion in SMN2 messenger ribonucleic acid (mRNA) transcripts in a human subject having mutations in the SMN1 gene that lead to functional SMN protein deficiency, in a human subject in need thereof. The method involves administering by an injection into the intrathecal space of the human subject a CNS delivery composition wherein the antisense oligonucleotide consists of 18 linked nucleosides, wherein the oligonucleotide has a nucleobase sequence consisting of the nucleobase sequence ^{Me}U^{Me}CA^{Me}C^{Me}U^{Me}U^{Me}U^{Me}CA^{Me}UAA^{Me}UG^{Me}C^{Me}UGG (SEQ ID NO:1), wherein each internucleoside linkage of the oligonucleotide is a phosphorothioate linkage, each nucleoside of the oligonucleotide is a 2′-methoxyethyl nucleoside, ^{Me}U is a 5-methyl-uracil, and ^{Me C} is a 5-methylcytosine.

In some embodiments, the injection is a bolus injection.

In another aspect, the disclosure relates to a method for delivering an antisense oligonucleotide to the CNS of a human subject. The method involves administering by intrathecal injection the antisense oligonucleotide encapsulated within a PLGA nanoparticle wherein the lactic acid:glycolic acid ratio of the PLGA nanoparticle is in the range of 2:98 to 100:0, and wherein the antisense oligonucleotide is complexed with PEI or another cationic molecule (e.g., a cationic peptide, chitosan, hexadecylamine, lauric arginate).

In certain instances, the PLGA nanoparticles comprise lactic acid:glycolic acid in a ratio selected from the group consisting of: 2:98, 3:97, 4:96, 5:95, 6:94, 7:93, 8:92, 9:91, 10:90, 11:89, 12:88, 13:87, 14:86, 15:85, 16:84, 17:83, 18:82, 19:81, 20:80, 21:79, 22:78, 23:77, 24:76, 25:75, 26:74, 27:73, 28:72, 29:71, 30:70, 31:69, 32:68, 33:67, 34:66, 35:65, 36:64, 37:63, 38:62, 39:61, 40:60, 41:59, 42:58, 43:57, 44:56, 45:55, 46:54, 47:53, 48:52, 49:51, 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:4, 98:2, and 100:0.

In certain instances, the human subject has a CNS disorder. In some instances, the CNS disorder is a synucleinopathy or a tauopathy. In some cases, the CNS disorder is SMA, ALS, Parkinson's disease, Alzheimer's disease, Huntington's disease, Angelman syndrome, frontotemporal dementia (FTD), Creutzfeldt-Jakob disease, spinocerebellar ataxia type 3 (SCA3), or Menkes disease.

In some instances, the antisense oligonucleotide is delivered to the CNS (e.g., cortex, striatum, thalamus, substantia nigra, cerebellum) of the human subject about 0.1 hours to about 1 week after administration. In some instances, the antisense oligonucleotide is delivered to the CNS (e.g., cortex, striatum, thalamus, substantia nigra, cerebellum) of the human subject 1 day to 7 days after administration. In some instances, the antisense oligonucleotide is delivered to the CNS (e.g., cortex, striatum, thalamus, substantia nigra, cerebellum) of the human subject 1 day to 6 days after administration. In some instances, the antisense oligonucleotide is delivered to the CNS (e.g., cortex, striatum, thalamus, substantia nigra, cerebellum) of the human subject 1 day to 5 days after administration. In some instances, the antisense oligonucleotide is delivered to the CNS (e.g., cortex, striatum, thalamus, substantia nigra, cerebellum) of the human subject 1 day to 4 days after administration. In some instances, the antisense oligonucleotide is delivered to the CNS (e.g., cortex, striatum, thalamus, substantia nigra, cerebellum) of the human subject 1 day to 3 days after administration. In some instances, the antisense oligonucleotide is delivered to the CNS (e.g., cortex, striatum, thalamus, substantia nigra, cerebellum) of the human subject 1 day to 2 days after administration. In some instances, the antisense oligonucleotide is delivered to the CNS (e.g., cortex, striatum, thalamus, substantia nigra, cerebellum) of the human subject 1 day after administration. In some instances, the antisense oligonucleotide is delivered to the cortex, striatum, thalamus, substantia nigra, cerebellum of the human subject within 0.1 hours to 48 hours after administration. In some instances, the antisense oligonucleotide is delivered to the cortex, striatum, thalamus, substantia nigra, cerebellum of the human subject within 0.1 hours to 36 hours after administration. In some instances, the antisense oligonucleotide is delivered to the cortex, striatum, thalamus, substantia nigra, cerebellum of the human subject within 0.1 hours to 24 hours after administration. In some instances, the antisense oligonucleotide is delivered to the cortex, striatum, thalamus, substantia nigra, cerebellum of the human subject within 0.1 hours to 12 hours after administration. In some instances, the antisense oligonucleotide is delivered to the cortex, striatum, thalamus, substantia nigra, cerebellum of the human subject within 0.1 hours to 6 hours after administration. In some instances, the antisense oligonucleotide is delivered to the cortex, striatum, thalamus, substantia nigra, cerebellum of the human subject within 0.1 hours to 3 hours after administration. In some instances, the antisense oligonucleotide is delivered to the cortex, striatum, thalamus, substantia nigra, cerebellum of the human subject within 0.1 hours to 2 hours after administration. In some instances, the antisense oligonucleotide is delivered to the cortex, striatum, thalamus, substantia nigra, cerebellum of the human subject within 24 hours after administration. In some instances, the antisense oligonucleotide is delivered to the cortex, striatum, thalamus, substantia nigra, cerebellum of the human subject within 12 hours after administration. In some instances, the antisense oligonucleotide is delivered to the cortex, striatum, thalamus, substantia nigra, cerebellum of the human subject within 6 hours after administration. In some instances, the antisense oligonucleotide is delivered to the cortex, striatum, thalamus, substantia nigra, cerebellum of the human subject within 3 hours after administration. In some instances, the antisense oligonucleotide is delivered to the cortex, striatum, thalamus, substantia nigra, cerebellum of the human subject within 2 hours after administration. In some instances, the antisense oligonucleotide is delivered to the cortex, striatum, thalamus, substantia nigra, cerebellum of the human subject within 1 hour after administration.

In another aspect, the disclosure provides a method of increasing the amount of an antisense oligonucleotide delivered to the spinal cord and/or brain of a human subject in need thereof relative to delivery of a solution of the antisense oligonucleotide in an aqueous buffer. The method involves intrathecally injecting a PLGA nanoparticle that encapsulates the antisense oligonucleotide, wherein the antisense oligonucleotide is pre-complexed with PEI or another cationic molecule (e.g., a cationic peptide, chitosan, hexadecylamine, lauric arginate), and wherein the lactic acid:glycolic acid ratio of the PLGA nanoparticle is in the range of 2:98 to 100:0. In certain instances, the PLGA nanoparticles comprise lactic acid:glycolic acid in a ratio selected from the group consisting of: 2:98, 3:97, 4:96, 5:95, 6:94, 7:93, 8:92, 9:91, 10:90, 11:89, 12:88, 13:87, 14:86, 15:85, 16:84, 17:83, 18:82, 19:81, 20:80, 21:79, 22:78, 23:77, 24:76, 25:75, 26:74, 27:73, 28:72, 29:71, 30:70, 31:69, 32:68, 33:67, 34:66, 35:65, 36:64, 37:63, 38:62, 39:61, 40:60, 41:59, 42:58, 43:57, 44:56, 45:55, 46:54, 47:53, 48:52, 49:51, 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:4, 98:2, and 100:0. In one instance, the PLGA nanoparticle comprises lactic acid:glycolic acid in a ratio of 50:50. In another instance, the PLGA nanoparticle comprises lactic acid:glycolic acid in a ratio of 5:95. In certain instances, the ASO comprises or consists of a nucleic acid sequence set forth in SEQ ID NO: 1. In certain instances, the ASO comprises or consists of a nucleic acid sequence that is useful to treat a neurodegenerative disease.

In another aspect, the disclosure provides a method of delivering an antisense oligonucleotide deeper into the brain of the human subject relative to delivery of a solution of the antisense oligonucleotide in an aqueous buffer. The method comprises intrathecally injecting a PLGA nanoparticle that encapsulates the antisense oligonucleotide, wherein the antisense oligonucleotide is pre-complexed with PEI or another cationic molecule (e.g., a cationic peptide, chitosan, hexadecylamine, lauric arginate), and wherein the lactic acid:glycolic acid ratio of the PLGA nanoparticle is in the range of 2:98 to 100:0. In certain instances, the PLGA nanoparticles comprise lactic acid:glycolic acid in a ratio selected from the group consisting of: 2:98, 3:97, 4:96, 5:95, 6:94, 7:93, 8:92, 9:91, 10:90, 11:89, 12:88, 13:87, 14:86, 15:85, 16:84, 17:83, 18:82, 19:81, 20:80, 21:79, 22:78, 23:77, 24:76, 25:75, 26:74, 27:73, 28:72, 29:71, 30:70, 31:69, 32:68, 33:67, 34:66, 35:65, 36:64, 37:63, 38:62, 39:61, 40:60, 41:59, 42:58, 43:57, 44:56, 45:55, 46:54, 47:53, 48:52, 49:51, 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:4, 98:2, and 100:0. In one instance, the PLGA nanoparticle comprises lactic acid:glycolic acid in a ratio of 50:50. In another instance, the PLGA nanoparticle comprises lactic acid:glycolic acid in a ratio of 5:95. In certain instances, the ASO comprises or consists of a nucleic acid sequence set forth in SEQ ID NO: 1. In certain instances, the ASO comprises or consists of a nucleic acid sequence that is useful to treat a neurodegenerative disease.

In certain instances, more of the antisense oligonucleotide is delivered to the striatum, thalamus, substantia nigra, and/or cerebellum of the brain relative to delivery of a solution of the antisense oligonucleotide in an aqueous buffer. The efficient delivery and distribution of the antisense oligonucleotide can result in reducing the number of administrations and improve patient experience and compliance.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1 shows the drug release profile of PLGA nanoparticles prepared with scaled-up ASO loading at a flow rate of 8 mL/min over 2 hr.

FIG. 2 depicts the knockdown levels from ICV injection of nanoparticles in mice.

FIG. 3 shows the knockdown in the spinal cord following intrathecal (IT) injection of nanoparticle formulations.

FIG. 4 illustrates the knockdown in cortex and striatum following IT injection of nanoparticle formulations.

FIG. 5 represents the release profile of ASO from PLGA nanoparticles and the free ASO over 48 hrs.

FIG. 6 illustrates the knockdown in the spinal cord, cerebellum, cortex, and striatum following T injection of nanoparticle formulations.

FIG. 7 depicts the knockdown in spinal cord, cerebellum, cortex and striatum following IT injection of nanoparticle formulations.

FIG. 8 shows the luciferase intensity from the brain after ICV injection of nanoparticle formulations in the reporter mice model.

### DETAILED DESCRIPTION

Delivery of drugs to the central nervous system (CNS) has been a central problem in the treatment of neurological diseases as the blood brain barrier (BBB) provides an effective barricade preventing most therapeutic molecules from entering the brain. Without access to the brain, the therapeutic effect of these molecules can be compromised or eliminated. Intrathecal (IT) injection provides a way to directly access the CNS and bypass the BBB. However, because the cerebrospinal fluid (CSF) into which the therapeutic is injected during IT administration is turned over several times a day, the residence time of a therapeutic is often limited. Without sufficient exposure to the CNS, the therapeutic benefit of the treatment can be limited. To increase the efficacy of IT administered therapeutics, Applicant attempted to improve the distribution of the oligonucleotides to target tissues within the CNS and therefore extend the exposure time and prevent fast clearance of the drug by improving the distribution of the oligonucleotide within the CNS. Applicant reasoned that in this manner a greater amount of the therapeutic can reach the targeted regions in the brain for longer time. Applicant attempted to improve distribution by encapsulating the therapeutic in a nanoparticle which will interact with tissues in a manner that is distinct from the negatively charged free oligonucleotide, have a longer residence time in the CNS and release the therapeutic of interest from the nanoparticle during the time it is present in the CNS.

Polymeric nanoparticles (e.g., PLGA nanoparticles) are usually designed to have a slow and controlled release kinetics and the skilled person in the art designing these drug delivery systems should match the release profile to the therapeutic needs for each particular indication. Most polymeric nanoparticles release their contents over extended periods of time (hours to months) to maximize residence time and control delivery of the drug. This disclosure, however, is based on the benefit of employing nanoparticles that release ASO rather quickly (hours to days) due to the biological limitation of CNS delivery and fast turnover of CSF. Applicant finds that one can get more therapeutic agent (e.g., ASO) along the spinal column and into the deeper regions of the brain, by encapsulating the oligonucleotide in a nanoparticle that has a relatively short half-life to ensure releasing the drug before clearance. To achieve this, Applicant's approach is to design the nanoparticle release rates that allow the particle to travel from the site of injection into the lumbar region of the spine to the brain and then release their contents before the particles get cleared from the CSF. These rates are much faster than those typically employed for extended release nanoparticle formulations. Encapsulating the drug inside the nanoparticles will increase the retention time in the CSF; however, the nanoparticles will not remain there permanently until they are completely broken down like most other polymeric nanoparticle (e.g., PLGA nanoparticle) therapies are designed to do. Applicant finds that these polymeric nanoparticles are still small enough that they will be eliminated from the intrathecal space around the CNS, just not as quickly as the free ASO. Thus, Applicant refrains from developing a polymeric nanoparticle (e.g., a PLGA nanoparticle) that releases for a very long time. Rather, Applicant relies on polymeric nanoparticles (e.g., PLGA nanoparticles) that release on the order of hours to days to a week (not several weeks to months) to make sure the therapeutic agent (e.g., ASO) is released and available while the particles still have access to the central nervous system and the brain.

This disclosure is also based, in part, on the finding that administration of nanoparticles to the central nervous system does not have toxic effects, and direct administration of Applicant's polymeric nanoparticles containing antisense oligonucleotide (ASO) to the central nervous system do not show adverse effects.

This disclosure provides, inter alfa, results from animal studies investigating the efficacy and safety of poly (dl-lactide-co-glycolide) (PLGA) nanoparticles for the delivery of antisense oligonucleotides (ASO) to the brain. Briefly, PLGA nanoparticles were loaded with Malat-1 ASO and characterized. These particles were then injected intracerebroventricularly (ICV) into mice. These injections were well-tolerated and showed knockdown in the lumbar spinal cortex indicating that the ASOs were released from the nanoparticles and the nanoparticles were safe at the doses used in these studies. Subsequently, nanoparticles were injected by intrathecal injection into rats. Results from this study show that ASOs administered encapsulated in nanoparticles result in a 1.5 to 2 fold increase in knockdown in the cortex as compared to free unformulated ASO alone.

Although there was an improvement from the particle formulations compared to free ASO, more work was needed to examine the performance differences among the PLGA formulations with different lactic acid:glycolic acid ratios. After refinement of the nanoparticle manufacturing process, improvements were applied to supply nanoparticles with different lactic acid:glycolic acid ratios for additional rat IT studies that examined tolerability of the formulations and distribution of the oligonucleotides along the spinal column and to the brain. Results demonstrated that there was an increase in the knockdown in the cortex from the nanoparticles (PLGA 50:50) when compared to free, unformulated ASO. In further ICV administration in the reporter mouse model with live in vivo imaging showed that the PLGA formulations resulted in significant improvement compared to the corresponding free unformulated ASO.

### Polymeric Nanocarrier Compositions

There are two broad approaches for delivering nucleic acids (e.g., oligonucleotide) to a target site in the body. The first is to chemically modify the nucleic acid, usually with a targeting ligand, while preserving the molecular nature of the conjugate. The second is to incorporate the nucleic acid into some form of nanocarrier that then determines the tissue distribution and cellular interactions of the oligonucleotide. The major distinction between these two approaches lies in the size of the delivery moiety: molecular scale versus nanoscale. This disclosure focuses on nanoscale delivery. There are various nanoscale systems including lipid nanocarriers and polymeric nanocarriers. This disclosure relates to polymeric nanocarriers. Polymeric nanocarriers include, for example, PLGA nanoparticles, polymeric micelles (also known as “core-shell” nanoparticles), a self-assembled hybrid nanocarrier comprised of a PLGA core and a lipid-PEG shell, and a nanohydrogel (e.g., the PRINT nanohydrogel).

Nanoparticles are considered one of the most versatile drug delivery systems, as they are able to protect therapeutic agents while efficiently delivering them into the target tissue or organ. Several different types of polymeric nanocarriers can be used for delivery of a therapeutic agent (e.g., oligonucleotide such as an ASO). In certain instances, the polymeric nanoparticles is one of poly(l-lactide), poly(glycolide), poly(d, l-lactide) (PLA), poly(dioxanone), poly(d, l-lactide-co-l-lactide), poly(d, l-lactide-co-glycolide), poly(glycolide-co-trimethylene carbonate), poly(caprolactone) (“polycaprolactone”), poly(d, l-lactide-co-glycolide) (PLGA), poly(dioxanone) poly(glycolide-co-trimethylene carbonate), or mixtures thereof. Exemplary lactic acid polymers are described for example in EP1468035, U.S. Pat. No. 6,706,854, WO2007/009919A2, EP1907023A, EP2263707A, EP2147036, EP0427185 and U.S. Pat. No. 5,610,266.

The biodegradable polymer PLGA has immense potential as a carrier for drug delivery. Additionally, it is possible to tune the overall physical properties of the PLGA-drug matrix by controlling parameters such as polymer molecular weight, ratio of lactide to glycolide, and the particle size to achieve desired drug loading and release rate. In aqueous environments, PLGA degrades by the hydrolysis of its ester linkages. Because of this, the hydrophobicity and crystallinity of a polymer impact its degradation rate: the more hydrophobic and the more crystalline a polymer, the slower it degrades. Of PLGA's two monomers, LA is more hydrophobic, so the more LA present in a PLGA polymer, the more hydrophobic it is. Also, the more LA present in a PLGA polymer, the more crystalline it is; the combination of these two characteristics make PLGA polymers with higher LA contents have slower degradation rates and those with higher GA contents have faster degradation rates. This feature of PLGA is useful and can help determine the most effective polymer type to choose for a desired release rate.

In some instances, the disclosure features PLGA nanoparticles comprising lactic acid:glycolic acid in a ratio in the range of 2:98 to 100:0. In certain instances, the disclosure features PLGA nanoparticles comprising lactic acid:glycolic acid in a ratio in the range of 2:98 to 98:2. In some instances, the disclosure features PLGA nanoparticles comprising lactic acid:glycolic acid in a ratio in the range of 5:95 to 95:5. In other instances, the disclosure features PLGA nanoparticles comprising lactic acid:glycolic acid in a ratio in the range of 10:90 to 90:10. In other instances, the disclosure features PLGA nanoparticles comprising lactic acid:glycolic acid in a ratio in the range of 15:85 to 85:15. In still other instances, the disclosure features PLGA nanoparticles comprising lactic acid:glycolic acid in a ratio in the range of 20:80 to 80:20. In some instances, the disclosure features PLGA nanoparticles comprising lactic acid:glycolic acid in a ratio in the range of 25:75 to 75:25. In other instances, the disclosure features PLGA nanoparticles comprising lactic acid:glycolic acid in a ratio in the range of 30:70 to 70:30. In certain instances, the disclosure features PLGA nanoparticles comprising lactic acid:glycolic acid in a ratio in the range of 35:65 to 65:35. In some instances, the disclosure features PLGA nanoparticles comprising lactic acid:glycolic acid in a ratio in the range of 40:55 to 55:45. In some instances, the disclosure features PLGA nanoparticles comprising lactic acid:glycolic acid in a ratio in the range of 5:95 to 85:15. In certain instances, the disclosure features PLGA nanoparticles comprising lactic acid:glycolic acid in a ratio selected from the group consisting of: 2:98, 3:97, 4:96, 5:95, 6:94, 7:93, 8:92, 9:91, 10:90, 11:89, 12:88, 13:87, 14:86, 15:85, 16:84, 17:83, 18:82, 19:81, 20:80, 21:79, 22:78, 23:77, 24:76, 25:75, 26:74, 27:73, 28:72, 29:71, 30:70, 31:69, 32:68, 33:67, 34:66, 35:65, 36:64, 37:63, 38:62, 39:61, 40:60, 41:59, 42:58, 43:57, 44:56, 45:55, 46:54, 47:53, 48:52, 49:51, 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:4, 98:2, and 100:0. In one instance, the disclosure features PLGA nanoparticles comprising lactic acid:glycolic acid in a ratio of 50:50. In another instance, the disclosure features PLGA nanoparticles comprising lactic acid:glycolic acid in a ratio of 5:95. In yet another instance, the disclosure features PLGA nanoparticles comprising lactic acid:glycolic acid in a ratio of 85:15.

In some instances, the polymeric nanocarrier (e.g., a PLGA nanoparticle) has an overall charge density of −0.3 to −12.0 mV. In other instances, the polymeric nanocarrier (e.g., a PLGA nanoparticle) has an overall charge density of −0.4 to −10.0 mV. In some instances, the polymeric nanocarrier (e.g., a PLGA nanoparticle) has an overall charge density of −0.4 to −1.0 mV. In some instances, the polymeric nanocarrier (e.g., a PLGA nanoparticle) has an overall charge density of −0.4 to −0.9 mV. In some instances, the polymeric nanocarrier (e.g., a PLGA nanoparticle) has an overall charge density of −0.4 to −0.8 mV. In some instances, the polymeric nanocarrier (e.g., a PLGA nanoparticle) has an overall charge density of −0.4 to −0.7 mV. In certain instances, the polymeric nanocarrier (e.g., a PLGA nanoparticle) has an overall charge density of −0.4 to −0.6 mV. In certain instances, the polymeric nanocarrier (e.g., a PLGA nanoparticle) has an overall charge density of −0.01 to −0.05 mV.

In some instances, the polymeric nanocarrier (e.g., a PLGA nanoparticle) has a polydispersity index of 0.2 to 0.9. In certain instances, the polymeric nanocarrier (e.g., a PLGA nanoparticle) has a polydispersity index of 0.2 to 0.8. In other instances, the polymeric nanocarrier (e.g., a PLGA nanoparticle) has a polydispersity index of 0.2 to 0.7. In some instances, the polymeric nanocarrier (e.g., a PLGA nanoparticle) has a polydispersity index of 0.2 to 0.6. In some instances, the polymeric nanocarrier (e.g., a PLGA nanoparticle) has a polydispersity index of 0.2 to 0.5. In some instances, the polymeric nanocarrier (e.g., a PLGA nanoparticle) has a polydispersity index of 0.2 to 0.4. In certain instances, the polymeric nanocarrier (e.g., a PLGA nanoparticle) has a polydispersity index of 0.2 to 0.3.

In some instances, the polymeric nanoparticle (e.g., PLGA nanoparticle) has a diameter of 100 nm to 1000 nm. In certain instances, the polymeric nanoparticle (e.g., PLGA nanoparticle) has a diameter of 100 nm to 900 nm. In some instances, the polymeric nanoparticle (e.g., PLGA nanoparticle) has a diameter of 100 nm to 800 nm. In certain instances, the polymeric nanoparticle (e.g., PLGA nanoparticle) has a diameter of 100 nm to 700 nm. In other instances, the polymeric nanoparticle (e.g., PLGA nanoparticle) has a diameter of 100 nm to 600 nm. In yet other instances, the polymeric nanoparticle (e.g., PLGA nanoparticle) has a diameter of 100 nm to 500 nm. In some instances, the polymeric nanoparticle (e.g., PLGA nanoparticle) has a diameter of 100 nm to 400 nm. In certain instances, the polymeric nanoparticle (e.g., PLGA nanoparticle) has a diameter of 100 nm to 300 nm. In some instances, the polymeric nanoparticle (e.g., PLGA nanoparticle) has a diameter of 100 nm to 250 nm. In other instances, the polymeric nanoparticle (e.g., PLGA nanoparticle) has a diameter of 100 nm to 200 nm.

In certain instances, the polymeric nanocarrier (e.g., a PLGA nanoparticle) has a diameter of 100 to 650 nM, an overall charge density of −0.4 to −0.6 mV, and a polydispersity index of 0.2 to 0.3. In certain instances, the polymeric nanocarrier (e.g., a PLGA nanoparticle) has a diameter of 100 to 400 nM, an overall charge density of −0.4 to −0.6 mV, and a polydispersity index of 0.2 to 0.3. In certain instances, the polymeric nanocarrier (e.g., a PLGA nanoparticle) has a diameter of 200 to 300 nM, an overall charge density of −0.4 to −0.6 mV, and a polydispersity index of 0.2 to 0.3. In certain instances, these polymeric nanocarriers (e.g., PLGA nanoparticles) comprise lactic acid:glycolic acid in a ratio selected from the group consisting of: 2:98, 3:97, 4:96, 5:95, 6:94, 7:93, 8:92, 9:91, 10:90, 11:89, 12:88, 13:87, 14:86, 15:85, 16:84, 17:83, 18:82, 19:81, 20:80, 21:79, 22:78, 23:77, 24:76, 25:75, 26:74, 27:73, 28:72, 29:71, 30:70, 31:69, 32:68, 33:67, 34:66, 35:65, 36:64, 37:63, 38:62, 39:61, 40:60, 41:59, 42:58, 43:57, 44:56, 45:55, 46:54, 47:53, 48:52, 49:51, 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:4, 98:2, and 100:0.

In certain instances, the negatively charged (anionic) oligonucleotide is complexed with a cationic molecule. Exemplary cationic molecules include a synthetic cationic polymer such as polyethylene imine (PEI), a natural cationic polymer such as chitosan, a cationic peptide, a cationic dendrimer, hexadecylamine, or lauric arginate. In some embodiments, the PEI can be a linear PEI or a cross-linked PEI. As PEIs are linear or branched polymers that have multiple titratable amino groups they can readily form nanocomplexes with oligonucleotides. In some embodiments, the complexing molecule can be PepFect6, a cationic peptide derived from bee melittin, a cationic peptide of the Transactivator of Transcription (TAT), a human lactoferrin-derived peptide, or a short amphipathic sequence. In some embodiments, the cationic dendrimer is a Polyamidoamine (PAMAM).

In some instances, the polymeric nanoparticle (e.g., PLGA nanoparticle) further comprises polyethylene glycol (PEG). A neutral polymer such as PEG can minimize protein binding and uptake by the reticuloendothelial system (RES). A polymeric nanoparticle comprising PEG can thus have increased circulation time. In certain instances, the PEG is linked to the polymeric nanocarrier with cleavable linkers or short lipid anchors. In some instances, the polymeric nanoparticle (e.g., PLGA nanoparticle) further comprises folate. The folate can be coupled to the nanocarrier surface. In some instances, the polymeric nanoparticle (e.g., PLGA nanoparticle) further comprises a small molecule ligand (e.g., anisamide) or an aptamer to target the site of interest. In some instances, the polymeric nanoparticle (e.g., PLGA nanoparticle) further comprises a transferrin receptor ligand. In some instances, the polymeric nanoparticle (e.g., PLGA nanoparticle) further comprises an anti-transferrin receptor antibody or fragment thereof. In some instances, the polymeric nanoparticle (e.g., PLGA nanoparticle) further comprises a rabies virus peptide to target the nanoparticles to neurons. In some instances, the polymeric nanoparticle (e.g., PLGA nanoparticle) further comprises a targeting ligand that targets a receptor site for endocytosis.

In certain instances, the polymeric nanoparticle (e.g., PLGA nanoparticle) comprises a therapeutic agent in addition to the nucleic acid (e.g., oligonucleotide such as an ASO) encapsulated within the nanoparticle. In certain cases, the therapeutic agent is selected from the group consisting of a small molecule, a cDNA, an mRNA, an siRNA, an miRNA, an aptamer, a ribozyme, and a different antisense oligonucleotide.

The polymeric nanoparticle (e.g., PLGA nanoparticle) can be prepared so that it encapsulates a nucleic acid (e.g., an oligonucleotide such as an ASO) to be delivered. In certain instances, the ASO comprises or consists of a sequence set forth in SEQ ID NO: 1. Such a polymeric nanoparticle can include a cationic molecule complexed with the nucleic acid (e.g., an oligonucleotide such as an ASO). In certain instances, the cationic molecule is PEI. In some instances, such polymeric nanocarriers can convey both a nucleic acid (e.g., an ASO) and a second therapeutic agent (e.g., a small molecule drug or another ASO). Thus, the polymeric nanoparticles can be employed for in vivo co-delivery of therapeutic agents to a target site (e.g., any part of the central nervous system such as the spinal cord, cortex, striatum, thalamus, substantia nigra, or cerebellum).

Any of the polymeric nanoparticles (e.g., PLGA nanoparticle) described above can be formulated for intrathecal delivery to a human subject. In some instances, the polymeric nanoparticles (e.g., PLGA nanoparticle) is administered intrathecally by a bolus injection. In some instances, the polymeric nanoparticle (e.g., PLGA nanoparticle) is formulated for delivery to one or more of the striatum, thalamus, substantia nigra, or cerebellum of the brain of a human subject.

### Antisense Oligonucleotides

Antisense oligonucleotides (ASOs) are synthetic single stranded strings of nucleic acids that bind to ribonucleic acid (RNA) and thereby alter or reduce expression of the target RNA. They can not only reduce expression of proteins by breakdown of the targeted transcript, but also restore protein expression or modify proteins through interference with pre-mRNA splicing. This disclosure encompasses ASOs of both types. In certain instances, the ASO of this disclosure is a “gapmer.” Such ASOs primarily act by selectively cleaving mRNAs that have complementary sites through an RNase H-dependent mechanism. They have a central region that supports RNase H activity flanked by chemically modified ends that increase affinity and/or reduce susceptibility to nucleases. In some instances, the ASO of this disclosure is a splice switching oligonucleotide (SSO) (e.g., nusinersen). SSOs are generally fully modified so as to ablate RNase H activity and allow interaction with nuclear pre-mRNA during splicing. They can be designed to bind to the 5′ or 3′ splice junctions or to exonic splicing enhancer or silencer sites. By binding to such sites they can modify splicing by, e.g., promoting alternative use of exons, exon exclusion, or exon inclusion.

Ideally, a synthetic oligonucleotide (e.g., ASO) of this disclosure should bind to a specific sequence on a target RNA transcript and be stable. Synthetic oligonucleotides (e.g., ASOs) are foreign to the cells into which they are introduced and thus they become targets for endogenous nucleases. For synthetic oligonucleotides to attain the level of persistence in a cell that would be needed for them to accomplish their tasks, they generally need to be protected from those endogenous nucleases. Synthetic oligonucleotides can be modified by any modification known in the art, including but not limited to modification of the phosphodiester backbone, modification at the ribose 2′OH group, and modification of the ribose ring and nucleoside base. For example, modification of the phosphate backbone can include phosphorothioate (PS) modification, where a non-bridging phosphate oxygen is replaced with sulfur. Additionally, other modifications include phosphorodithioates and phosphonoacetates. See e.g., U.S. Pat. Nos. 6,143,881, 5,587,361 and 5,599,797, which are incorporated by reference. Other modifications include 2′-O-methyl (2′OMe), 2′Fluoro (2′F), 2′Methoxyethyl (2′-O-MOE), 2′Fluorarabino (FANA), 2′-H, 2′-Thiouracil, locked nucleic acid (LNA), constrained Ethyl (cEt), bridged nucleic acid (BNA), ethylene-bridged nucleic acid (ENA), hexitol nucleic acid (HNA), altritol nucleic acid (ANA), cyclohexene nucleic acid (CeNA), unlocked nucleic acid (UNA), 4′Thio (4′-S), and 3′ inverted abasic end cap. In some embodiments, a nucleic acid may be modified by substituting a native phosphodiester linkage with a boranophosphate (PB) linkage, a phosphonoacetate (Pac) linkage or a thiophosphonoacetate backbone linkage. In some embodiments, the nucleic acid may include more than one modification. In some embodiments, the nucleic acid may comprise more than two modifications. In some cases, the modification of the synthetic oligonucleotides (e.g., ASO) is at least one of: a 2′-O-methyl (2′OMe) modification, a 2′Fluoro (2′F) modification, a MOE modification, a 2′Fluorarabino (FANA) modification, a 2′-H modification, a 2′-Thiouracil modification, a locked nucleic acid (LNA) modification, a bridged nucleic acid (BNA) modification, an ethylene-bridged nucleic acid (ENA) modification, a hexitol nucleic acid (HNA) modification, an altritol nucleic acid (ANA) modification, a cyclohexene nucleic acid (CeNA) modification, an unlocked nucleic acid (UNA) modification, a 4′Thio (4′-S) modification, a thiol linkage modification, and a 3′ inverted abasic end cap modification.

In some instances, the synthetic oligonucleotide (e.g., ASO) has one or more phosphorothioate (PS) backbone modifications. Such modifications improve stability and protection from nucleases in the blood and tissues. They also promote protein binding and thus support interactions with albumin and other blood proteins and in this manner retard renal clearance. This modification supports RNase H activity so can be used in both gapmers and SSOs. In certain instances, the ASO comprise a phosphorodiamidate morpholino oligomer (PMO) and/or peptide nucleic acid (PNA) modification. Such modifications create neutral backbones and offer high resistance to nucleases. As such modifications do not support RNase H activity, they are primarily used in SSOs rather than gapmers. Another modification is the alteration of at the 2′ sugar position. Such modifications include the 2′-O-Me and 2′-O-(2-methoxyethyl) (MOE) modifications. These modifications promote an RNA-like conformation and significantly increase binding affinity to RNA whilst also providing enhanced nuclease resistance. Oligonucleotides that are fully modified at the 2′ position do not support RNase H activity and so are generally best suited for SSOs. However, RNAse H dependent antisense effects can be achieved by using “gapmers” that contain a central unmodified region of about 7 residues flanked by 2′ modified region. Another modification that can be effective in oligonucleotides is the use of bridged rings. The locked nucleic acid (LNA) chemistry and the constrained ethyl (cEt) as well as the tricycle-DNA (tc-DNA) modifications involve bridging of the sugar ring. Such modifications promote an RNA-like structure, exhibit nuclease resistance, and provide dramatic increases in binding affinity. These modifications can be used in both gapmers and SSOs. In certain embodiments, the ASOs of this disclosure include one or more of the above-described modifications. In some embodiments, the ASOs of this disclosure have a PS backbone. In some embodiments, the ASOs of this disclosure have a mixed PS and phosphodiester backbone. In certain embodiments, the ASOs of this disclosure have one or more 2′-O-(2-methoxyethyl) (MOE) modifications. In certain embodiments, the ASOs of this disclosure, all residues have MOE modifications. In certain embodiments, the ASOs of this disclosure include one or more cEt. In certain instances, one or more uracils of the ASOs of this disclosure are replaced by 5-methyl-uracil. In certain instances, all uracils of the ASOs of this disclosure are replaced by 5-methyl-uracil. In certain instances, one or more cytosines of the ASOs of this disclosure are replaced by 5-methyl-cytosine. In certain instances, all cytosines of the ASOs of this disclosure are replaced by 5-methyl-cytosine.

Non-limiting examples of the ASOs that are encompassed by this disclosure are provided in Table I.

TABLE I
Exemplary Antisense Oligonucleotides

| Antisense Oligonucleotide Sequence | Design | Length |
|---|---|---|
| 5′-^{Me}U^{Me}CA^{Me}C^{Me}U^{Me}U^{Me}U^{Me}CA^{Me}UAA^{Me}UG^{Me}C^{Me}UGG-3′ | SSO | 18 |
| (SEQ ID NO: 1) | | |
| 5′-G^{Me}Co^{Me}CoAoGG^{Me}CTGGTTATGAo^{Me}Co^{Me}U^{Me}CA-3′ | 5-10-5 | 20 |
| (SEQ ID NO: 2) | gapmer | |
| 5′-G^{Me}C^{Me}UA^{Me}U^{Me}UA^{Me}C^{Me}C^{Me}U^{Me}UAA^{Me}C^{Me}C^{Me}CAG-3′ | SSO | 18 |
| (SEQ ID NO: 3) | | |

wherein: the underlined nucleoside has a 2′-O-(2-methoxyethyl) (MOE) modification; the “o” is a phosphodiester internucleoside linkage and the absence of “o” indicates a phosphorothioate internucleoside linkage; “^{Me}U” is 5-methyl-uracil; and “^{Me}C” is 5-methyl-cytosine.

Other non-limiting and exemplary ASOs encompassed by this disclosure are provided in Evers et al., Advanced Drug Delivery Reviews, 87:90-103 (2015) (see, e.g., Table 2 and the references cited therein); Bennett et al., Annu. Rev. Pharmacol. Toxicol., 61:831-52 (2021) (see, e.g., Table 1 and the references cited therein); Silva et al., Brain, 143; 407-429 (2020) (see, e.g., Table 2 and the references cited therein); U.S. Pat. Nos. 10,385,341; 9,683,235; and 10,407,680, the content of all of which are incorporated by reference herein in their entirety.

The ASOs described herein are encapsulated in a polymeric nanocarrier (e.g., PLGA). In certain instances, the ASOs described herein are complexed with a cationic molecule (e.g., PEI).

### Pharmaceutical Compositions

The polymeric nanoparticles disclosed herein may be combined with pharmaceutically acceptable carriers to form a pharmaceutical composition. As would be appreciated by one of skill in this art, the carriers may be chosen based on the route of administration as described below, the location of the target issue, the drug being delivered, the time course of delivery of the drug, etc.

Injectable preparations, for example, sterile injectable aqueous or oleaginous suspensions, may be formulated using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In one embodiment, the conjugate is suspended in a carrier fluid comprising 1% (w/v) sodium carboxymethyl cellulose and 0.1% (v/v) TWEEN™80. The injectable formulations can be sterilized, for example, by filtration through a bacteria-retaining filter, or by terminal sterilization of the solid compositions which can be dissolved or dispersed in sterile water or injectable diluent of choice prior to administration.

It will be appreciated that the exact dosage of a polymeric nanoparticle containing a nucleic acid agent (e.g., ASO) is chosen by the individual physician in view of the patient to be treated, in general, dosage and administration are adjusted to provide an effective amount of the nucleic acid agent nanoparticle to the patient being treated. As used herein, the “effective amount” of a nanoparticle containing a nucleic acid agent (e.g., an ASO) refers to the amount necessary to elicit the desired biological response. As will be appreciated by those of ordinary skill in this art, the effective amount of a polymeric nanoparticle (e.g., PLGA nanoparticle) containing a nucleic acid agent (e.g., ASO) may vary depending on such factors as the desired biological endpoint, the drug to be delivered, the target tissue, the route of administration, etc. For example, the effective amount of a polymeric nanoparticle containing a nucleic acid agent (e.g., ASO) might be the amount that results in a reduction in tumor size by a desired amount over a desired period of time. Additional factors that may be taken into account include the severity of the disease state; age; weight, and gender of the patient being treated; diet, time and frequency of administration; drug combinations; reaction sensitivities; and tolerance/response to therapy.

The polymeric nanoparticles of this disclosure may be formulated in dosage unit form for ease of administration and uniformity of dosage. The expression “dosage unit form” as used herein refers to a physically discrete unit of nanoparticle appropriate for the patient to be treated. It will be understood, however, that the total daily usage of the compositions will be decided by the attending physician within the scope of sound medical judgment. For any nanoparticle, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of nanoparticles can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions that exhibit large therapeutic indices may be useful in some embodiments. The data obtained from cell culture assays and animal studies can be used in formulating a range of dosage for human use.

In some embodiments, a composition suitable for freezing is contemplated, including polymeric nanoparticles disclosed herein and a solution suitable for freezing, e.g., a sugar such as a mono, di, or poly saccharide, e.g., sucrose and/or a trehalose, and/or a salt and/or a cyclodextrin solution is added to the nanoparticle suspension. The sugar (e.g., sucrose or trehalose) may act, e.g., as a cryoprotectant to prevent the particles from aggregating upon freezing. For example, provided herein is a nanoparticle formulation comprising a plurality of disclosed nanoparticles, sucrose, an ionic halide, and water; wherein the nanoparticles/sucrose/water/ionic halide is about 3-40%/10-40%/20-95%/0.1-10% (w/w/w/w) or about 5-10%/10-15%/80-90%/1-10% (w/w/w/w). For example, such solution may include nanoparticles as disclosed herein, about 5% to about 20% by weight sucrose and an ionic halide such as sodium chloride, in a concentration of about 10-100 mM. In another example, provided herein is a nanoparticle formulation comprising a plurality of disclosed nanoparticles, trehalose, cyclodextrin, and water; wherein the nanoparticles/trehalose/water/cyclodextrin is about 3-40%/1-25%/20-95%/1-25% (w/w/w/w) or about 5-10%/1 -25%/80-90%/10-15% (w/w/w/w).

### Methods of Delivery

This disclosure features methods of delivering a nucleic acid (e.g., an oligonucleotide such as an antisense oligonucleotide) to the central nervous system (CNS) of a human subject. Accessing the CNS is a difficult task because of the blood brain barrier (BBB). The BBB is comprised of tightly linked endothelial cells supported by a network of pericytes and astrocyte processes and is impervious to molecules as small as sucrose. The BBB is also largely impervious to oligonucleotides.

One way of dealing with this delivery problem is by direct administration of oligonucleotides. This can be done, e.g., by intrathecal injection. When administered intrathecally oligonucleotides distribute broadly in the CNS and are taken up by both neurons and glial cells.

In certain instances, the polymeric nanocarrier (e.g., PLGA nanoparticle) comprising a nucleic acid (e.g., antisense oligonucleotide) is administered to the human subject by intrathecal injection. In some cases, the intrathecal injection is a bolus injection. In certain embodiments, the antisense oligonucleotide is encapsulated within a PLGA nanoparticle. In some cases, the antisense oligonucleotide is complexed with a cationic molecule (e.g., PEI, chitosan, hexadecylamine, lauric arginate, or a cationic peptide). In some cases, the lactic acid:glycolic acid ratio of the PLGA nanoparticle is in the range of 2:98 to 98:2. In certain instances, the PLGA nanoparticle comprises lactic acid:glycolic acid in a ratio selected from the group consisting of: 2:98, 3:97, 4:96, 5:95, 6:94, 7:93, 8:92, 9:91, 10:90, 11:89, 12:88, 13:87, 14:86, 15:85, 16:84, 17:83, 18:82, 19:81, 20:80, 21:79, 22:78, 23:77, 24:76, 25:75, 26:74, 27:73, 28:72, 29:71, 30:70, 31:69, 32:68, 33:67, 34:66, 35:65, 36:64, 37:63, 38:62, 39:61, 40:60, 41:59, 42:58, 43:57, 44:56, 45:55, 46:54, 47:53, 48:52, 49:51, 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:4, 98:2, and 100:0.

This disclosure features the delivery of polymeric nanoparticle carriers that release their encapsulated cargo (e.g., an ASO) with “fast” release kinetics. By “fast” is meant release of the cargo within about 0.1 hours to about 1 week of the intrathecal injection of the polymeric nanoparticle carrier. In some embodiments, the cargo is released within 7 days, 6 days, 5 days, 4 days, 3 days, 2 days or 1 day of the intrathecal injection of the polymeric nanoparticle carrier. In other embodiments, the cargo is released within 0.1 hours, 0.2 hours, 0.3 hours, 0.4 hours, 0.5 hours, 0.6 hours, 0.7 hours, 0.8 hours, 0.9 hours, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, or 48 hours of the intrathecal injection of the polymeric nanoparticle carrier.

The compositions and delivery methods disclosed herein permit increasing the amount of a nucleic acid (e.g., an antisense oligonucleotide) delivered to the brain of a human subject relative free unformulated ASO delivery.

In addition, the compositions and delivery methods disclosed herein permit increasing the time that a delivered nucleic acid (e.g., an ASO) is present and active in the CNS of a human subject relative to unformulated ASO delivery.

Furthermore, the compositions and delivery methods disclosed herein allow for delivering a nucleic acid (e.g., an ASO) deeper into the brain of the human subject relative to unformulated ASO delivery. In some instances, more of the nucleic acid (e.g., an ASO) is delivered to the striatum, thalamus, substantia nigra, and/or cerebellum of the brain of a human subject relative to unformulated ASO delivery.

### Methods of Treatment

This disclosure features methods of treating a CNS disorder in a human subject in need thereof. The method involves administering to the subject a therapeutically effective amount of a polymeric nanocarrier composition described herein. In some instances, the CNS disorder is a synucleinopathy or a tauopathy. In certain instances, the CNS disorder is spinal muscular atrophy (SMA), amyotrophic lateral sclerosis (ALS), Parkinson's disease, Alzheimer's disease, Huntington's disease, Angelman syndrome, frontotemporal dementia (FTD), Creutzfeldt-Jakob disease, spinocerebellar ataxia type 3 (SCA3), or Menkes disease. In some instances, the polymeric nanocarrier comprises an antisense oligonucleotide comprising or consisting of the sequence set forth in SEQ ID NO:1. In some instances, the polymeric nanocarrier further comprises an additional therapeutic agent (e.g., a small molecule compound). In some instances, the polymeric nanocarrier comprises a dose of the ASO of between 0.05 mg/kg to 25 mg/kg. In some instances, the polymeric nanocarrier comprises a dose of the ASO of between 0.05 mg/kg to 20 mg/kg. In some instances, the polymeric nanocarrier comprises a dose of the ASO of between 0.05 mg/kg to 15 mg/kg. In some instances, the polymeric nanocarrier comprises a dose of the ASO of between 0.05 mg/kg to 10 mg/kg. In some instances, the polymeric nanocarrier comprises a dose of the ASO of between 0.05 mg/kg to 5 mg/kg. In some instances, the polymeric nanocarrier comprises a dose of the ASO of between 0.05 mg/kg to 4 mg/kg. In some instances, the polymeric nanocarrier comprises a dose of the ASO of between 0.05 mg/kg to 3 mg/kg. In some instances, the polymeric nanocarrier comprises a dose of the ASO of between 0.05 mg/kg to 2 mg/kg. In some instances, the polymeric nanocarrier comprises a dose of the ASO of between 0.05 mg/kg to 1 mg/kg. A therapeutically effective amount can be readily determined by a health care provider based, inter alfa, on the age, sex, and stage of disease of the human subject being treated. In some cases, the polymeric nanocarrier is administered by intrathecal (IT) injection. In certain instances, the IT injection is a bolus injection.

In one instance, the disclosure features a method of treating spinal muscular atrophy (SMA) in a human subject. In another instance, provided is a method of increasing inclusion of exon 7 in SMN2 messenger ribonucleic acid (mRNA) transcripts in a human subject having loss of both functional copies of the SMN1 gene. In yet another instance, featured is a method for increasing exon 7 inclusion in SMN2 messenger ribonucleic acid (mRNA) transcripts in a human subject having mutations in the SMN1 gene that lead to functional SMN protein deficiency. In one embodiment of these methods, the human subject is administered by an injection into the intrathecal space of the human subject a CNS delivery composition (e.g., a PLGA nanoparticle) comprising an ASO that can be used to treat SMA (e.g., nusinersen). In one embodiment, the antisense oligonucleotide that is encapsulated in the polymeric nanocarrier that is administered to the human subject comprises or consists of the nucleobase sequence ^{Me}U^{Me}CA^{Me}C^{Me}U^{Me}U^{Me}U^{Me}CA^{Me}UAA^{Me}UG^{Me}C^{Me}UGG (SEQ ID NO:1), wherein each internucleoside linkage of the oligonucleotide is a phosphorothioate linkage, each nucleoside of the oligonucleotide is a 2′-methoxyethyl nucleoside, ^{Me}U is a 5-methyl-uracil, and ^{Me}C is a 5-methylcytosine.

In certain instances, the intrathecal injection is by bolus injection.

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### EXAMPLES

### Example 1: Study 1—Materials & Methods

Study 1 is described in Examples 1-6.

### Materials

PLGA lactide:glycolide (50:50), ester terminated, average 100,000 Da
PLGA lactide:glycolide (85:15), ester terminated, 190,000-240,000 Da
PLGA L-lactide:glycolide (5:95), 190,000-210,000 Da
Polyethylenimine (PEI), linear, 2.5 kDa, Sigma 764604
Ethyl acetate
Malat-1 antisense oligonucleotide: G^{Me}Co^{Me}CoAoGG^{Me}CTGGTTATG Ao^{Me}Co^{Me}U^{Me}CA(SEQ ID NO:2)_(wherein “o” is phosphodiester (if not labeled with “o” phosphorothioate; ^{Me}U is 5-methyl-uracil; ^{Me}C is 5-methyl-cytosine; and the underlined nucleosides are MOE), 7 kDa
Phosphate buffered saline, 1X, pH 7.4, Life Technologies 10010023
Polyvinyl alcohol
Modified Polyethersulfone Hollow Fiber Filter Modules, 750 kDa cutoff
Water for injection
2% Brij S100 surfactant
Malic Acid Buffer, pH 3

### PLGA Nanoparticle Preparation Protocol

Malat-1 loaded poly (dl-lactide-co-glycolide) (PLGA) particles were prepared using the double emulsion solvent evaporation technique. Three different types of PLGA polymers—PLGA lactide:glycolide (50:50), PLGA lactide:glycolide (85:15), and PLGA lactide:glycolide (5:95)—were used to create nanoparticles. Because the three polymers differ in their lactic acid (LA) to glycolic acid (GA) ratio, the release rates of the antisense oligonucleotide (ASO) should vary, with the slowest releasing nanoparticles being a result of the having the highest LA content.

In short, a polyvinyl alcohol (PVA) solution was created by dissolving PVA in phosphate buffered saline (PBS). PLGA was dissolved in ethyl acetate. Polyethylenimine (PEI) was dissolved in water. The ASO was dissolved in PBS at pH 7.4. The ASO solution and PEI solutions were mixed together at a one-to-one molar ratio. The PEI is a positively charged polymer and is complexed to the negatively charged ASO to prevent charge repulsion and to allow encapsulation into the negatively charged PLGA. The ASO-PEI complex was then mixed with the PLGA solution and sonicated to create a water-in-oil emulsion. This emulsion was then added to the PVA solution, which acts as a stabilizer for the nanoparticles, and sonicated to create a water-in-oil-in-water emulsion. The ethyl acetate was then removed by evaporation. The emulsion was purified, and the buffer exchanged by tangential flow filtration (TFF) using PBS. The resulting emulsion was characterized by several analytic techniques to measure the size, polydispersity, and ASO loading of the particles and then stored at −20° C. until administration to animals.

### Example 2: Characterization of ASO Loaded PLGA Nanoparticles

The results presented here summarize the characterization of the nanoparticles made with each type of PLGA when Malat-1 ASO is encapsulated in the particles. The size distribution shown in Table 1 indicates that particles made with the 85:15 PLGA have the largest hydrodynamic diameter where particles made with the PLGA 50:50 have the smallest. The particles made with 85:15 and 5:95 PLGA were nearly neutral, while the particles made with 50:50 were slightly negative. The majority of the particles had a polydispersity (PDI) of less than 0.3, which indicates a narrow size distribution. Three measurements were made for each formulation, and the average reading is reported.

TABLE 1
Size distribution, polydispersity, and charge of PLGA
nanoparticle formulations containing Malat-1 ASO

| | | Polydispersity | |
|---|---|---|---|
| Sample | Diameter (nm) | index | Zeta potential (mV) |
| PLGA 85:15 | 437.7 | 0.23 | −0.27 |
| PLGA 50:50 | 355.1 | 0.34 | −11 |
| PLGA 5:95 | 389.9 | 0.28 | −0.69 |

### Example 3: Malat-1 ASO Concentration in the Nanoparticles

The concentration of Malat-1 ASO in the nanoparticles was determined by the UV absorbance at 260 nm after extracting the ASO from the particles. The UV readings and corresponding ASO concentrations are shown in Table 2.

TABLE 2
Loading capacity of PLGA formulations

| | Absorbance | | ASO concentration |
|---|---|---|---|
| Sample | at 260 nm | Dilution Factor | (mg/mL) |
| PLGA 85:15 | 0.1816 | 700X | 4.8 |
| PLGA 50:50 | 0.3216 | 500X | 5.8 |
| PLGA 5:95 | 0.6514 | 200X | 4.7 |

### Example 4: In Vitro Release of ASO From PLGA Nanoparticles

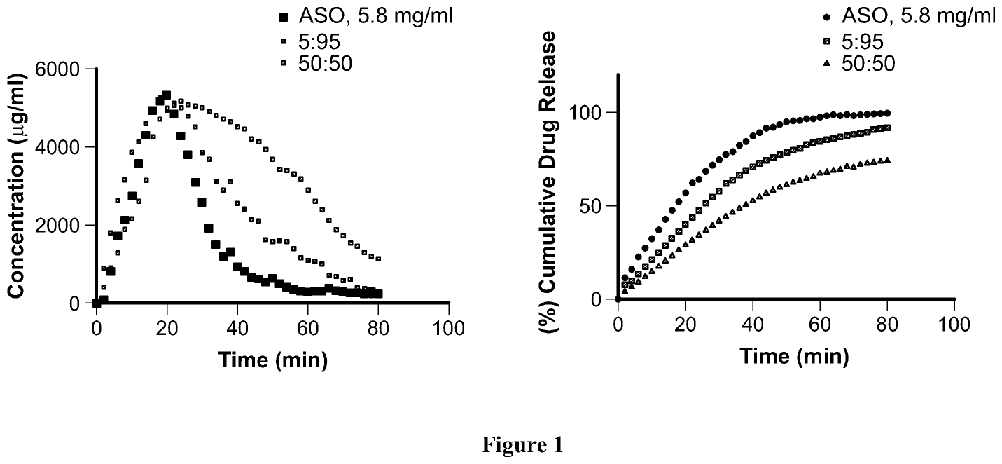

The in vitro release kinetics of ASO loaded nanoparticles were investigated using particles made from three different PLGA compositions, 5:95, 50:50 and 85:15. The release kinetics were performed on the SOTAX USB IV dissolution apparatus in an open system. Mechanical and chemical properties, swelling behavior, resistance to hydrolysis and subsequently biodegradation rate of the polymer are directly influenced by the degree of crystallinity of the PLGA, which is further dependent on the molar ratio of the individual monomer components in the copolymer chain. Crystalline PGA, when co-polymerized with PLA, reduces the degree of crystallinity of PLGA and as a result increase the rate of hydrolysis and degradation. Results for the release of ASO from two groups of nanoparticles can be seen in FIG. 1. Free ASO (not encapsulated in a nanoparticle) at a 6 mg/mL concentration was used as a control. Using an 8 mL/min flow rate, the nanoparticles made with 5:95 PLGA were found to have the fastest release followed by 50:50 and 85:15.

### Example 5: Delivery of PLGA Nanoparticles Intracerebroventricularly in Mice

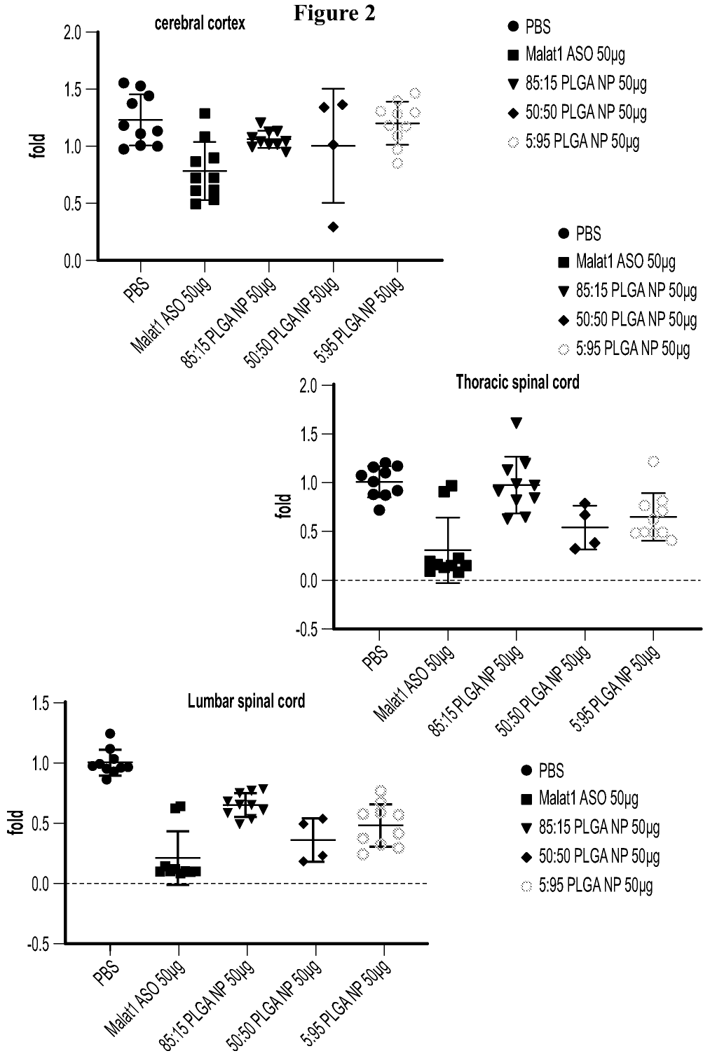

An efficacy study comparing the effects of unformulated Malat-1 ASO vs. nanoparticle-encapsulated Malat-1 in C57B6 mice was performed by injecting the solutions intracerebroventricularly (ICV) into the mice. Ten mice were injected in each of five different groups (50 mice total): a buffer PBS control group, unformulated Malat-1 ASO groups (50 μg), and three PLGA nanoparticle groups described by their LA:GA ratio 85:15, 50:50, and 5:95 containing approximately 50 μg Malat1 ASO. Animals were followed for 1 week after ICV injection. Formulations were well tolerated with no observable safety signals. Knockdown levels are shown for various areas of the CNS in FIG. 2.

In mice injected with the nanoparticle formulations, significant knockdown was observed in the lumbar spinal cord region. This indicates that the Malat-1 ASO was released from the nanoparticles and able to act on the intended target. The animals dosed with the 50:50 LA:GA ratio nanoparticles showed higher knockdown compare to 85:15 and 5:95 PLGA nanoparticles specially in the lumbar spinal cortex and indicating that the ASOs were released from 50:50 nanoparticles had better distribution and provided more effective delivery compare to other nanoparticle groups.

### Example 6: Delivery of PLGA Nanoparticles Intrathecally in Rats

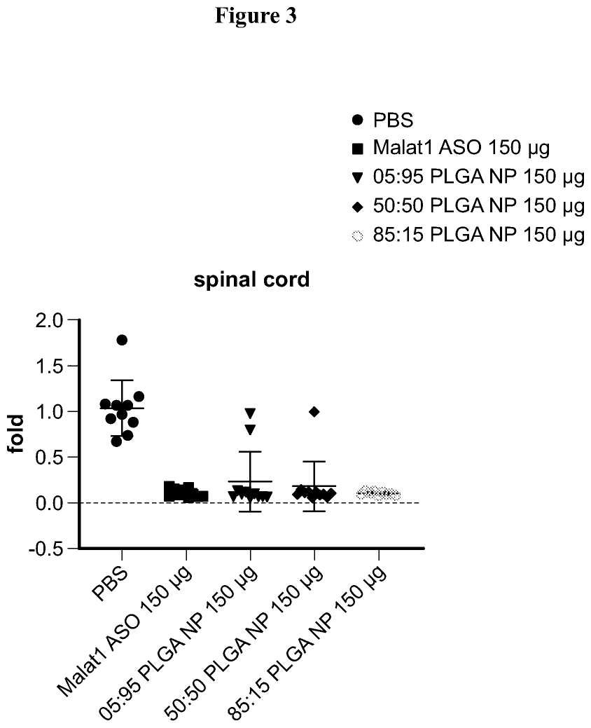

To test the nanoparticle delivery of Malat-1 ASO in the intrathecal (IT) space, the same formulations were prepared and injected intrathecally in rats. A similar experimental design as the mouse ICV study in Example 5 was used for this rat study. A total of 50 rats were used in this experiment, 10 rats in each of five dose cohorts: a buffer PBS control group, unformulated Malat-1 ASO group (150 μg) and three PLGA nanoparticle groups, 85:15, 50:50, and 5:95. Rats were sacrificed after 2 weeks, and the percent knockdown of the Malat-1 expression in various regions of the CNS and brain was measured. No safety issues were observed in rats after injecting the solutions. A signal knockdown of greater than 90% in the spinal cord region of all groups (FIG. 3) indicates that the injections were performed well.

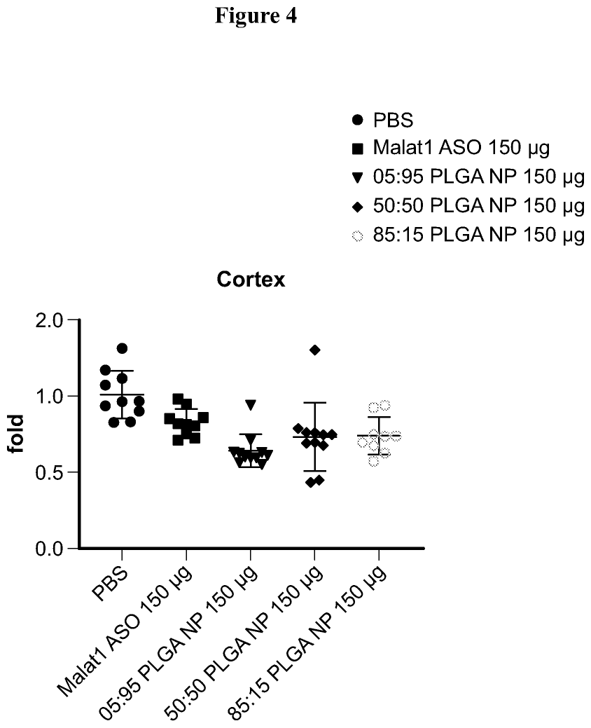

Malat-1 expression was also measured in different regions of the brain, specifically the cerebral cortex (FIG. 4). This data demonstrates that the ASO was delivered from the PLGA particles and reached the deeper regions of the brain such as the striatum. Also, this experiment indicated that administration via IT injection is safe.

In conclusion, ASO encapsulated nanoparticles with three different release rates were made and injected by ICV injection into mice. Knockdown in these mice demonstrated that the ASOs were released from the nanoparticles and were still active. Additionally, no safety signals were noted. These same formulations were injected intrathecally into rats. Knockdown in these rats further demonstrated that the ASOs can be released from the nanoparticles and reach deeper regions of the brain.

### Example 7: Study 2—Materials & Methods

Study 2 is described in Examples 7-11.

### Materials

PLGA lactide:glycolide (50:50), ester terminated, average 100,000 Da
PLGA lactide:glycolide (85:15), ester terminated, 190,000-240,000 Da
PLGA L-lactide:glycolide (5:95), 190,000-210,000 Da
Polyethylenimine (PEI), linear, 2.5 kDa, Sigma 764604
Ethyl acetate
Malat-1 antisense oligonucleotide: : G^{Me}Co^{Me}CoAoGG^{Me}CTGGTTATG Ao^{Me}Co^{Me}U^{Me}CA(SEQ ID NO:2) (wherein “o” is phosphodiester (if not labeled with “o” phosphorothioate; ^{Me}U is 5-methyl-uracil; ^{Me}C is 5-methyl-cytosine; and the underlined nucleosides are MOE), 7 kDa
Phosphate buffered saline, 1X, pH 7.4, Life Technologies 10010023
Polyvinyl Alcohol
Modified Polyethersulfone Hollow Fiber Filter Modules, 750 kDa cutoff
Water for injection

### PLGA Nanoparticle Preparation Protocol

Malat-1 ASO loaded poly (dl-lactide-co-glycolide) (PLGA) particles were prepared using the double emulsion solvent evaporation technique. Three different types of PLGA polymers—PLGA lactide:glycolide (50:50), PLGA lactide:glycolide (85:15), and PLGA lactide:glycolide (5:95) were used to create nanoparticles.

Same protocol in Example 1 was followed with an enhanced pre-complexation step on ASO-PEI conjugation. Briefly, PLGA polymer was dissolved in ethyl acetate and mixed with a PEI-ASO pre-complex to form a water-in-oil emulsion. Prior to making the primary emulsion PEI was dissolved in deionized water by heating it up to 80° C. and mixing at 300 rpm. The temperature of the dissolved PEI solution was dropped to 60° C. to form the ASO-PEI pre-complex. The water-in-oil emulsion was further emulsified with a 2.5% w/v PVA solution to form the water-in-oil-water emulsion. The final emulsion was stirred for 18 hours at ambient condition to remove the solvent. The final product was purified, and buffer exchanged by tangential flow filtration (TFF) using PBS. These resulting nanoparticles were characterized by several analytic techniques to measure the size, polydispersity, and ASO loading and then stored frozen at −20° C. until administration to animals.

### Example 8: Characterization of ASO Loaded PLGA Nanoparticles

The particles were all less than 300 nm with PDI values below 0.25, indicating monodisperse size distributions (see, Table 3). The particles with a 5:95 LA:GA ratio had the smallest hydrodynamic diameter (176 nm), and particles with an 85:15 LA:GA ratio had the largest particle size (288.6 nm). Nearly neutral charge was observed for all three formulations.

TABLE 3
Size distribution, polydispersity, and charge of PLGA
nanoparticle formulations containing Malat-1 ASO

| | | Polydispersity | |
|---|---|---|---|
| Sample | Diameter (nm) | index | Zeta potential (mV) |
| PLGA 85:15 | 288.6 | 0.24 | 0.04 |
| PLGA 50:50 | 236.9 | 0.16 | −0.05 |
| PLGA 5:95 | 176 | 0.11 | −0.21 |

### Example 9: ASO Loading in the Nanoparticles

The amount of Malat-1 ASO concentration in the nanoparticles was determined by extracting the ASO from the nanoparticles and measuring the UV absorbance at 260 nm on a SoloVPE. The measured ASO concentrations are shown in Table 4.

TABLE 4
Malat-1 Concentration in PLGA formulations

| | ASO Concentration |
|---|---|
| Sample | (mg/mL) |
| PLGA 5:95 | 2.27 |
| PLGA 50:50 | 2.26 |
| PLGA 85:15 | 2.28 |

### Example 10: In Vitro Release of ASO From PLGA Nanoparticles

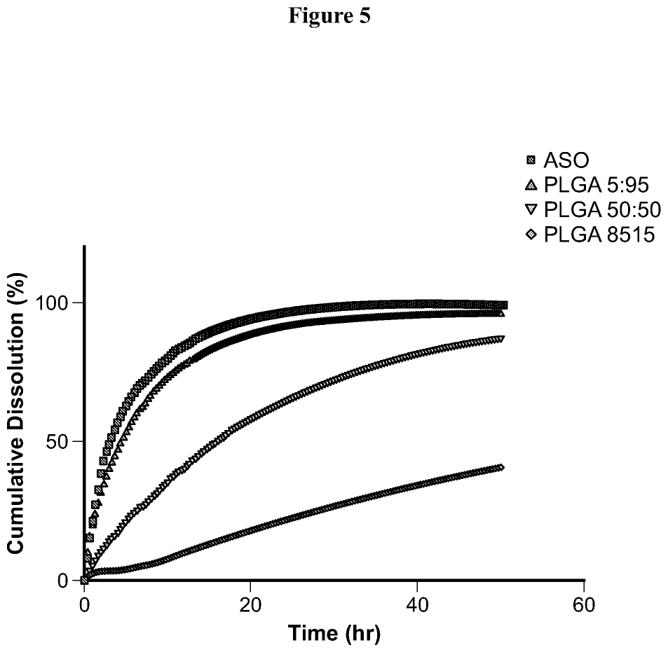

The in vitro release kinetics of ASO loaded nanoparticles were measured with a fully automated flow-through cell dissolution apparatus (USP 4, Sotax) in a closed-loop configuration. Cells with an internal diameter of 22.4 mm were used. A UV spectrophotometer at a wavelength of 260 nm was used to measure the ASO concentration of the dissolution fluid in real-time. The temperature was maintained at 37° C. during the process. The flow rate of the PBS dissolution medium through the cells was 16 mL/min. The dissolution apparatus pumped and re-circulated PBS through the cells, and as ASO was released from the nanoparticles, the change in concentration was detected. From this, cumulative release curves were created and can be seen in FIG. 5. Unencapsulated ASO at a 2.9 mg/mL concentration was used as a control. Nanoparticles made with 5:95 LA:GA ratio were found to have the fastest release. This is expected as this polymer has the lowest LA concentration, and therefore is the least crystalline and hydrophobic. The nanoparticles made with a ratio of 85:15 had the slowest release due to their higher content of glycolic acid and higher hydrophilicity.

### Example 11: Delivery of PLGA Nanoparticles Intrathecally in Rats

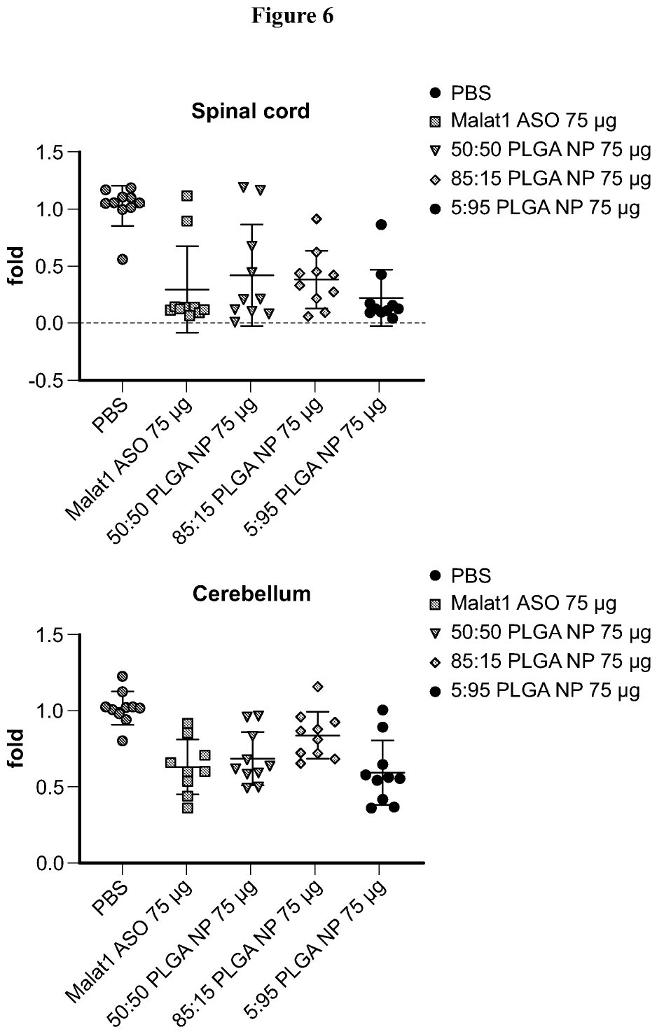

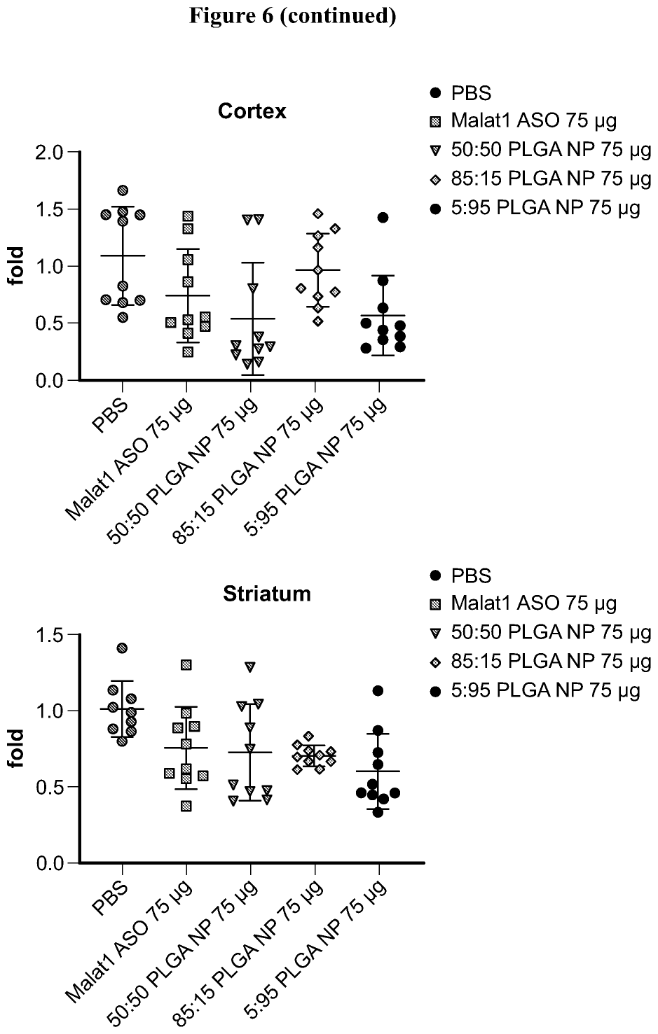

The gene modulation effects of Malat-1 ASO loaded PLGA nanoparticles were tested in Sprague-Dawley rats by injecting the solutions intrathecally and measuring knockdown of the Malat-1 gene in different sections of brain tissue. Ten rats were injected in each of five different groups including a buffer PBS control group, unformulated freeMalat-1 groups (75 μg), and the three nanoparticle groups as described above, 85:15, 50:50, 5:95, all containing approximately 75 μg Malat-1. Animals were monitored for 2 weeks after the IT injection. Formulations were well tolerated with no observable safety issues. Knockdown levels are shown for various areas of the CNS in FIG. 6. A knockdown of greater than 90% in the spinal cord region of all groups (FIG. 6) indicates that the injections were performed well, as nearly complete knockdown in this region is expected with successful injections. The Malat-1 knockdown in the cortex and striatum indicate that the ASO was released from the particles as they were distributed through the CNS (FIG. 6). The PLGA particles with the 85:15 composition showed lower knockdown percentages compared to the 50:50 and the 5:95. There was an increase in the knockdown from the 5:95 and 50:50 particles compared to the control dose of unformulated ASO.

In conclusion, Malat-1 ASO encapsulated PLGA nanoparticles with different release rates were administered intrathecally into rats. Particles with fast and medium release rates showed improved knockdown compared to unformulated ASOs. In order to confirm the effect of particle degradation rate on ASO knock-down in the CNS another rat IT study was tested.

### Example 12: Study 3—Materials & Methods

Study 3 is described in Examples 12-15.

### Materials

PLGA lactide:glycolide (50:50), ester terminated, average 40,000 Da
PLGA lactide:glycolide (75:25), ester terminated, 40,000 Da
PLA L-lactide, 40,000 Da
Polyethylenimine (PEI), linear, 2.5 kDa, Sigma 764604
Ethyl acetate
Malat-1 antisense oligonucleotide: : G^{Me}Co^{Me}CoAoGG^{Me}CTGGTTATG Ao^{Me}Co^{Me}U^{Me}CA(SEQ ID NO:2) (wherein “o” is phosphodiester (if not labeled with “o” phosphorothioate; ^{Me}U is 5-methyl-uracil; ^{Me}C is 5-methyl-cytosine; and the underlined nucleosides are MOE), 7 kDa
Phosphate buffered saline, 1X, pH 7.4, Life Technologies 10010023
Brij S100%
Modified Polyethersulfone Hollow Fiber Filter Modules, 500 kDa cutoff
Water for injection
%10 Sucrose

### PLGA Nanoparticle Preparation Protocol

In this study, Malat-1 ASO loaded PLGA nanoparticles were prepared using the double emulsion solvent evaporation technique. Three different types of PLGA polymers—PLGA lactide:glycolide (50:50), PLGA lactide:glycolide (75:15), and PLA lactide:glycolide (100:0) were used. As described previously, PLGA polymer was dissolved in ethyl acetate and mixed with a PEI-ASO (2.8:1) pre-complex to form a water-in-oil emulsion. The water-in-oil emulsion was further emulsified with a 0.2% w/v Brij S100% solution to form the water-in-oil-water emulsion. The final emulsion was stirred for 18 hours at ambient condition to remove the solvent. The final product was purified, and buffer exchanged by tangential flow filtration (TFF) using PBS and subsequently added into %10 sucrose formulation to prevent any non-specific aggregation during freeze/thaw cycle. These resulting nanoparticles were characterized by several analytic techniques to measure the size, polydispersity, and ASO loading and then stored frozen at −20° C. until administration to animals.

### Example 13: Characterization of ASO Encapsulated PLGA Nanoparticles

Particle sizes were consistent for each batch and were uniformly distributed as the PDI was less than 0.2 (Table 5). PLGA 75:25 composition yielded negative charge compared to other formulations.

TABLE 5
Size distribution, polydispersity, and charge of PLGA
nanoparticle formulations containing Malat-1 ASO

| | | Polydispersity | |
|---|---|---|---|
| Sample | Diameter (nm) | index | Zeta potential (mV) |
| PLGA 75:25 | 236 | 0.14 | −9.88 |
| PLGA 50:50 | 175 | 0.13 | −2.91 |
| PLA | 223 | 0.15 | −1.27 |

### Example 14: ASO Concentration in the Nanoparticles

The amount of Malat-1 ASO concentration in the nanoparticles was determined by extracting the ASO from the nanoparticles and measuring the UV absorbance at 260 nm on a SoloVPE. The measured ASO concentrations are shown in Table 6.

TABLE 6
Malat-1 Concentration in PLGA formulations

| | ASO Concentration |
|---|---|
| Sample | (mg/mL) |
| PLGA 75:25 | 2.63 |
| PLGA 50:50 | 2.73 |
| PLA | 2.65 |

### Example 15: Delivery of PLGA Nanoparticles Intrathecally in Rats

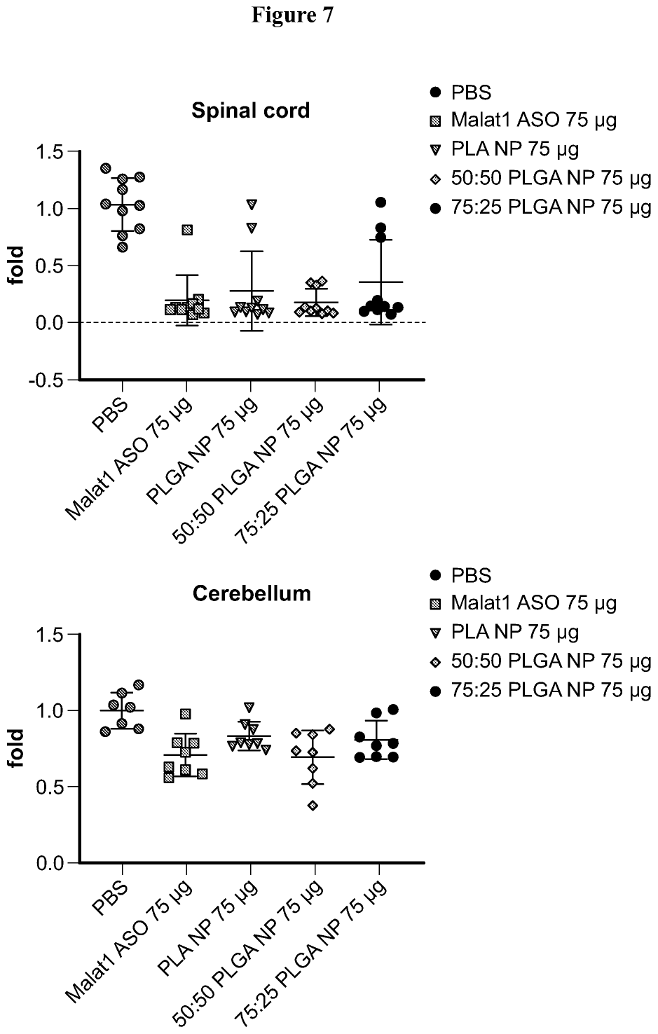

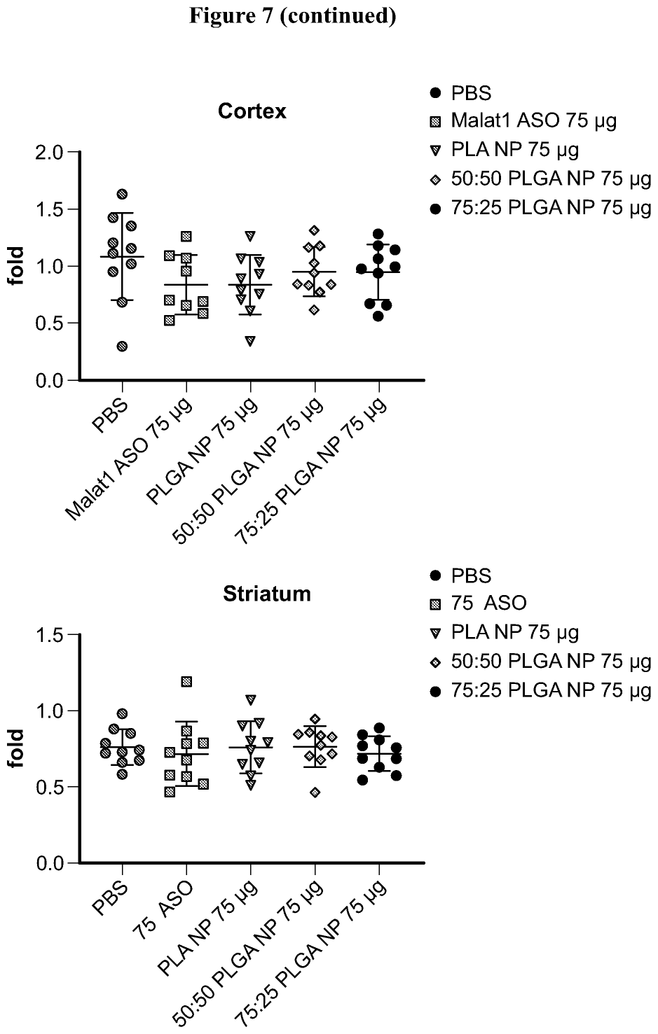

The knockdown effect of Malat-1 loaded PLGA nanoparticles were tested in Sprague-Dawley rats by injecting the solutions intrathecally and measuring knockdown of the Malat-1 gene in different sections of brain tissue. Ten rats were injected in each of five different groups including a buffer PBS control group, free Malat-1 group (75 μg), and the three nanoparticle groups as described above, 100:0, 75:25, 50:50, all containing approximately 75 μg Malat-1. Animals were kept alive for 2 weeks after the IT injection. Formulations were well tolerated with no observable safety issues. Knockdown levels are shown for various areas of the CNS in FIG. 7. A knockdown of greater than 90% in the spinal cord region of all groups (FIG. 7) indicates that the injections were performed well, as nearly complete knockdown in this region is expected with successful injections. The Malat-1 knockdown in the cortex and striatum indicate that the ASO was released from the particles as they were distributed through the CNS (FIG. 7). All nanoparticle formulations showed similar knock-down rate in the cortex and striatum compared to correspondent unformulated ASO. The results indicated that the PLGA formulations provided at least similar treatment when compared to unformulated ASO. PLGA 50:50 nanoparticles show even slightly higher efficacy compare to unformulated ASO and the other nanoparticle formulations. This study also confirmed the safe delivery of PLGA nanoparticles via IT administration. The current intrathecal administration method in rodents only show a single time-point to assess the knock-down level in CNS. Thus, in the next study we investigated the impact of PLGA nanoparticle by using a real-time imaging method to determine the ASO uptake in the CNS tissues.

### Example 16: Study 4—Materials & Methods

Study 4 is described in Examples 16-19.

### Materials

PLGA lactide:glycolide (50:50), ester terminated, average 200,000 Da
Polyethylenimine (PEI), linear, 2.5 kDa, Sigma 764604
Ethyl acetateβ-globin antisense oligonucleotide:G^{Me}C^{Me}UA^{Me}U^{Me}UA^{Me}C^{Me}C^{Me}UAA^{Me}C^{Me}C^{Me}CAG (SEQ ID NO:3) (wherein: the underlined nucleoside has a 2′-O-(2-methoxyethyl) (MOE) modification), 7 kDa
Phosphate buffered saline, 1X, pH 7.4, Life Technologies 10010023
Brij S100%
Modified Polyethersulfone Hollow Fiber Filter Modules, 500 kDa cutoff
%10 Sucrose

### PLGA Nanoparticle Preparation Protocol

β-globin ASO loaded PLGA nanoparticles were prepared using PLGA lactide:glycolide (50:50) with double emulsion solvent evaporation technique. Pre-complexed β-globin ASO-PEI solution was mixed in 2% Brij S100 surfactant followed by 60 second sonication at 100% and 60 second sonication at 80%. Secondary emulsion was created by using Malic Acid Buffer at pH 3 to enhance the stability of pre-complexed media in the primary emulsion in order to achieve target ASO load in the PLGA particles. Then, pH of the final solution was adjusted to 7.2 by diluting in PBS. The suspension was characterized by several analytic techniques to measure the size, polydispersity, and ASO loading of the particles and then stored at −20° C. prior to use in animals.

### Example 17: Characterization of (β-Globin ASO Encapsulated PLGA Nanoparticles

Particle size of (β-globin ASO loaded PLGA nanoparticles was measured using the same method that was previously used for Malat-1 formulations. The results indicated that (β-globin loaded nanoparticles are around 264 nm with uniformly distributed PDI value of 0.19.

### Example 18: β-globin ASO Concentration in the Nanoparticles

The concentration of (β-globin encapsulated in the nanoparticles was determined by extracting the ASO from the nanoparticles and measuring the UV absorbance at 260 nm on a SoloVPE. The measured (β-globin ASO concentration was 3.3 mg/mL.

### Example 19: In Vivo Live Imaging in Reporter Mice Model to Assess ASO Uptake

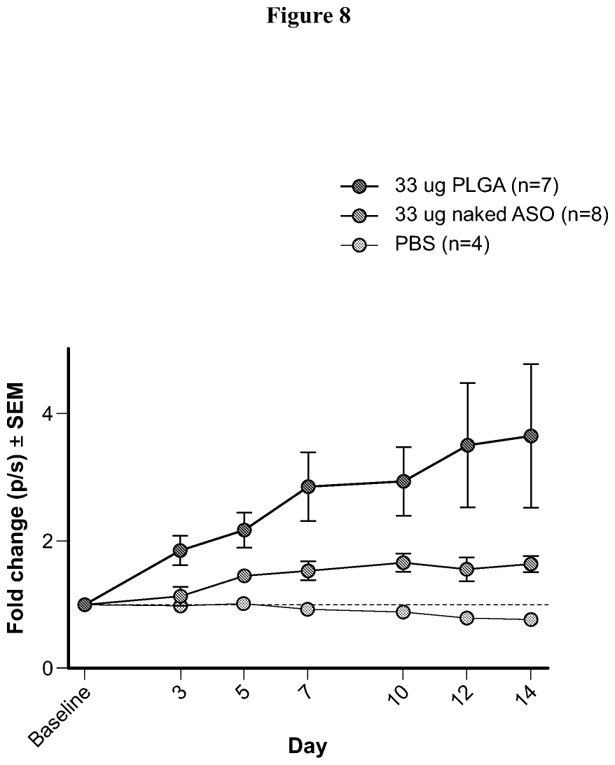

The potency gains of ASO loaded nanoparticles were investigated using a luciferase reporter mouse model. AAV reporter construct was designed to read out splice correction by bioluminescence imaging. The luciferase reporter gene in the construct is split by the beta-globin intron, which is spliced out in the presence of beta-globin ASO, resulting in the expression of luciferase gene. The AAV construct was administered through IV injection at postnatal day 0 to achieve a broad expression of AAV in the brain. In 6-8-week-old mice, ASO formulations were injected ICV at a dose equivalent to 33 μg ASO, and bioluminescence imaging above the head was taken at multiple time points using an IVIS imager. Each image was taken 10 minutes after an intraperitoneal injection of D-luciferin substrate. FIG. 8 shows the fold changes in bioluminescence from the baseline value before ASO administration. The bioluminescence signal in the animals dosed with encapsulated (β-globin is significantly higher than those administered by unformulated ASO at any given time point during 2 weeks imaging study. The results confirm that PLGA nanoparticles were able to provide faster and higher ASO uptake in the brain over a long period of time.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A central nervous system (CNS) delivery composition comprising a polymeric nanocarrier and an antisense oligonucleotide, wherein the antisense oligonucleotide is encapsulated within the polymeric nanocarrier, and wherein the antisense oligonucleotide is directly pre-complexed with a cationic molecule.

2. The CNS delivery composition of claim 1, wherein the polymeric nanocarrier is selected from the group consisting of poly(l-lactide), poly(glycolide), poly(d, l-lactide) (PLA), poly(dioxanone), poly(d, l-lactide-co-l-lactide), poly(d, l-lactide-co-glycolide), poly(glycolide-co-trimethylene carbonate), poly(caprolactone) (“polycaprolactone”), poly(d, l-lactide-co-glycolide) (PLGA), poly(dioxanone) poly(glycolide-co-trimethylene carbonate), and mixtures thereof.

3. The CNS delivery composition of claim 1, wherein the polymeric nanocarrier is PLGA.

4. The CNS delivery composition of claim 3, wherein the composition is a PLGA nanoparticle comprising lactic acid:glycolic acid in a ratio in the range of 2:98 to 100:0.

5. The CNS delivery composition of any one of claims 1 to 4, wherein the cationic molecule is a cationic peptide.

6. The CNS delivery composition of any one of claims 1 to 4, wherein the cationic molecule is a chitosan, hexadecylamine, or lauric arginate.

7. The CNS delivery composition of any one of claims 1 to 5, wherein the cationic molecule is a polyethylene imine (PEI).

8. The CNS delivery composition of claim 7, wherein the PEI is a linear PEI or a cross-linked PEI.

9. The CNS delivery composition of any one of claims 1 to 8, further comprising a therapeutic agent.

10. The CNS delivery composition of claim 9, wherein the therapeutic agent is selected from the group consisting of a small molecule, a cDNA, an mRNA, an siRNA, an miRNA, an aptamer, and a ribozyme.

11. The CNS delivery composition of any one of claims 1 to 10, formulated for intrathecal delivery to a human subject.

12. The CNS delivery composition of any one of claims 1 to 11, wherein the antisense oligonucleotide is a gapmer or a splice switching antisense oligonucleotide.

13. The CNS delivery composition of any one of claims 1 to 11, wherein the antisense oligonucleotide consists of a nucleic sequence set forth in SEQ ID NO:1.

14. The CNS delivery composition of any one of claims 1 to 11, wherein the antisense oligonucleotide consists of 18 linked nucleosides, wherein the oligonucleotide has a nucleobase sequence consisting of the nucleobase sequence ^{Me}U^{Me}CA^{Me}C^{Me}U^{Me}U^{Me}U^{Me}CA^{Me}UAA^{Me}UG^{Me}C^{Me}UGG (SEQ ID NO:1), wherein each internucleoside linkage of the oligonucleotide is a phosphorothioate linkage, each nucleoside of the oligonucleotide is a 2′-methoxyethyl nucleoside, ^{Me}U is a 5-methyl-uracil, and ^{Me}C is a 5-methylcytosine.

15. A method of treating a CNS disorder in a human subject in need thereof, the method comprising administering to the human subject a therapeutically effective amount of the CNS delivery composition of any one of claims 1 to 14.

16. The method of claim 15, wherein the administering is by intrathecal injection.

17. A method of treating spinal muscular atrophy (SMA), increasing inclusion of exon 7 in SMN2 messenger ribonucleic acid (mRNA) transcripts in a human subject having loss of both functional copies of the SMN1 gene, or increasing exon 7 inclusion in SMN2 messenger ribonucleic acid (mRNA) transcripts in a human subject having mutations in the SMN1 gene that lead to functional SMN protein deficiency, in a human subject in need thereof, the method comprising administering by an injection into the intrathecal space of the human subject the CNS delivery composition of claim 14.

18. The method of any one of claims 15 to 17, wherein the injection is a bolus injection.

19. A method for delivering an antisense oligonucleotide to the CNS of a human subject, the method comprising administering by intrathecal injection the antisense oligonucleotide encapsulated within a PLGA nanoparticle wherein the lactic acid:glycolic acid ratio of the PLGA nanoparticle is in the range of 2:98 to 100:0, and wherein the antisense oligonucleotide is pre-complexed with PEI, or another cationic molecule.

20. The method of claim 19, wherein the human subject has a CNS disorder.

21. The method of claim 20, wherein the CNS disorder is SMA, ALS, Parkinson's disease, Alzheimer's disease, Huntington's disease, Angelman syndrome, frontotemporal dementia (FTD), Creutzfeldt-Jakob disease, spinocerebellar ataxia type 3 (SCA3), Menkes disease, a synucleinopathy, or a tauopathy.

22. The method of any one of claims 19 to 21, wherein the antisense oligonucleotide is delivered to the CNS of the human subject 0.1 hours to 1 week after administration.

23. The method of any one of claims 19 to 21, wherein the antisense oligonucleotide is delivered to the CNS of the human subject 1 day to 6 days after administration.

24. The method of any one of claims 19 to 21, wherein the antisense oligonucleotide is delivered to the cortex of the human subject within 0.1 hours to 48 hours after administration.

25. The method of any one of claims 19 to 21, wherein the antisense oligonucleotide is delivered to the striatum of the human subject within 6 hours after administration.

26. The method of any one of claims 19 to 25, wherein the other cationic molecule is a cationic peptide, chitosan, hexadecylamine, or lauric arginate.

27. The method of any one of claims 19 to 25, wherein the PEI is a linear PEI or a cross-linked PEI.

28. A method of increasing the amount of an antisense oligonucleotide delivered to the brain of a human subject in need thereof relative to delivery of a solution of the antisense oligonucleotide in an aqueous buffer, the method comprising intrathecally injecting a PLGA nanoparticle that encapsulates the antisense oligonucleotide, wherein the antisense oligonucleotide is pre-complexed with PEI or another cationic molecule, and wherein the lactic acid:glycolic acid ratio of the PLGA nanoparticle is in the range of 2:98 to 100:0.

29. A method of delivering an antisense oligonucleotide deeper into the brain of the human subject relative to a solution of the antisense oligonucleotide in an aqueous buffer, the method comprising intrathecally injecting a PLGA nanoparticle that encapsulates the antisense oligonucleotide, wherein the antisense oligonucleotide is pre-complexed with PEI or another cationic molecule, and wherein the lactic acid:glycolic acid ratio of the PLGA nanoparticle is in the range of 2:98 to 100:0.

30. The method of claim 29, wherein more of the antisense oligonucleotide is delivered to the striatum, thalamus, substantia nigra, and/or cerebellum of the brain relative to the solution of the antisense oligonucleotide in the aqueous buffer.

31. A method of reducing the number of administrations of an antisense oligonucleotide into the spinal cord and/or brain of a human subject relative to a solution of the antisense oligonucleotide in an aqueous buffer, the method comprising intrathecally injecting a PLGA nanoparticle that encapsulates the antisense oligonucleotide, wherein the antisense oligonucleotide is pre-complexed with PEI or another cationic molecule, and wherein the lactic acid:glycolic acid ratio of the PLGA nanoparticle is in the range of 2:98 to 100:0.

32. The method of any one of claims 28 to 31, wherein the PEI is a linear PEI or a cross-linked PEI.

33. The method of any one of claims 28 to 31, wherein the another cationic molecule is a cationic peptide, chitosan, hexadecylamine, or lauric arginate.


SEQUENCE LISTING (3 sequences)

SEQ ID NO: 1 - RNA, 18 nt, Artificial Sequence
  source: /note=“Description of Artificial Sequence Synthetic oligonucleotide”
  modified_base (1)..(1): 2′-O-(2-methoxyethyl) 5-methyl-uracil
  misc_feature (1)..(2): /note=“Phosphorothioate internucleoside linkage”
  modified_base (2)..(2): 2′-O-(2-methoxyethyl) 5-methyl-cytosine
  misc_feature (2)..(3): /note=“Phosphorothioate internucleoside linkage”
  modified_base (3)..(3): 2′-O-(2-methoxyethyl) adenine
  misc_feature (3)..(4): /note=“Phosphorothioate internucleoside linkage”
  modified_base (4)..(4): 2′-O-(2-methoxyethyl) 5-methyl-cytosine
  misc_feature (4)..(5): /note=“Phosphorothioate internucleoside linkage”
  modified_base (5)..(5): 2′-O-(2-methoxyethyl) 5-methyl-uracil
  misc_feature (5)..(6): /note=“Phosphorothioate internucleoside linkage”
  modified_base (6)..(6): 2′-O-(2-methoxyethyl) 5-methyl-uracil
  misc_feature (6)..(7): /note=“Phosphorothioate internucleoside linkage”
  modified_base (7)..(7): 2′-O-(2-methoxyethyl) 5-methyl-uracil
  misc_feature (7)..(8): /note=“Phosphorothioate internucleoside linkage”
  modified_base (8)..(8): 2′-O-(2-methoxyethyl) 5-methyl-cytosine
  misc_feature (8)..(9): /note=“Phosphorothioate internucleoside linkage”
  modified_base (9)..(9): 2′-O-(2-methoxyethyl) adenine
  misc_feature (9)..(10): /note=“Phosphorothioate internucleoside linkage”
  modified_base (10)..(10): 2′-O-(2-methoxyethyl) 5-methyl-uracil
  misc_feature (10)..(11): /note=“Phosphorothioate internucleoside linkage”
  modified_base (11)..(11): 2′-O-(2-methoxyethyl) adenine
  misc_feature (11)..(12): /note=“Phosphorothioate internucleoside linkage”
  modified_base (12)..(12): 2′-O-(2-methoxyethyl) adenine
  misc_feature (12)..(13): /note=“Phosphorothioate internucleoside linkage”
  modified_base (13)..(13): 2′-O-(2-methoxyethyl) 5-methyl-uracil
  misc_feature (13)..(14): /note=“Phosphorothioate internucleoside linkage”
  modified_base (14)..(14): 2′-O-(2-methoxyethyl) guanine
  misc_feature (14)..(15): /note=“Phosphorothioate internucleoside linkage”
  modified_base (15)..(15): 2′-O-(2-methoxyethyl) 5-methyl-cytosine
  misc_feature (15)..(16): /note=“Phosphorothioate internucleoside linkage”
  modified_base (16)..(16): 2′-O-(2-methoxyethyl) 5-methyl-uracil
  misc_feature (16)..(17): /note=“Phosphorothioate internucleoside linkage”
  modified_base (17)..(17): 2′-O-(2-methoxyethyl) guanine
  misc_feature (17)..(18): /note=“Phosphorothioate internucleoside linkage”
  modified_base (18)..(18): 2′-O-(2-methoxyethyl) guanine
ucacuuucauaaugcugg

SEQ ID NO: 2 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
  modified_base (1)..(1): 2′-O-(2-methoxyethyl) guanine
  misc_feature (1)..(2): /note=“Phosphorothioate internucleoside linkage”
  modified_base (2)..(2): 2′-O-(2-methoxyethyl) 5-methyl-cytosine
  misc_feature (2)..(3): /note=“Phosphodiester internucleoside linkage”
  modified_base (3)..(3): 2′-O-(2-methoxyethyl) 5-methyl-cytosine
  misc_feature (3)..(4): /note=“Phosphodiester internucleoside linkage”
  modified_base (4)..(4): 2′-O-(2-methoxyethyl) adenine
  misc_feature (4)..(5): /note=“Phosphodiester internucleoside linkage”
  modified_base (5)..(5): 2′-O-(2-methoxyethyl) guanine
  misc_feature (5)..(6): /note=“Phosphorothioate internucleoside linkage”
  misc_feature (6)..(7): /note=“Phosphorothioate internucleoside linkage”
  modified_base (7)..(7): 5-methyl-cytosine
  misc_feature (7)..(8): /note=“Phosphorothioate internucleoside linkage”
  misc_feature (8)..(9): /note=“Phosphorothioate internucleoside linkage”
  misc_feature (9)..(10): /note=“Phosphorothioate internucleoside linkage”
  misc_feature (10)..(11): /note=“Phosphorothioate internucleoside linkage”
  misc_feature (11)..(12): /note=“Phosphorothioate internucleoside linkage”
  misc_feature (12)..(13): /note=“Phosphorothioate internucleoside linkage”
  misc_feature (13)..(14): /note=“Phosphorothioate internucleoside linkage”
  misc_feature (14)..(15): /note=“Phosphorothioate internucleoside linkage”
  misc_feature (15)..(16): /note=“Phosphorothioate internucleoside linkage”
  modified_base (16)..(16): 2′-O-(2-methoxyethyl) adenine
  misc_feature (16)..(17): /note=“Phosphodiester internucleoside linkage”
  modified_base (17)..(17): 2′-O-(2-methoxyethyl) 5-methyl-cytosine
  misc_feature (17)..(18): /note=“Phosphodiester internucleoside linkage”
  modified_base (18)..(18): 2′-O-(2-methoxyethyl) 5-methyl-uracil
  misc_feature (18)..(19): /note=“Phosphorothioate internucleoside linkage”
  modified_base (19)..(19): 2′-O-(2-methoxyethyl) 5-methyl-cytosine
  misc_feature (19)..(20): /note=“Phosphorothioate internucleoside linkage”
  modified_base (20)..(20): 2′-O-(2-methoxyethyl) adenine
gccaggctggttatgacuca

SEQ ID NO: 3 - RNA, 18 nt, Artificial Sequence
  source: /note=“Description of Artificial Sequence Synthetic oligonucleotide”
  modified_base (1)..(1): 2′-O-(2-methoxyethyl) guanine
  misc_feature (1)..(2): /note=“Phosphorothioate internucleoside linkage”
  modified_base (2)..(2): 2′-O-(2-methoxyethyl) 5-methyl-cytosine
  misc_feature (2)..(3): /note=“Phosphorothioate internucleoside linkage”
  modified_base (3)..(3): 2′-O-(2-methoxyethyl) 5-methyl-uracil
  misc_feature (3)..(4): /note=“Phosphorothioate internucleoside linkage”
  modified_base (4)..(4): 2′-O-(2-methoxyethyl) adenine
  misc_feature (4)..(5): /note=“Phosphorothioate internucleoside linkage”
  modified_base (5)..(5): 2′-O-(2-methoxyethyl) 5-methyl-uracil
  misc_feature (5)..(6): /note=“Phosphorothioate internucleoside linkage”
  modified_base (6)..(6): 2′-O-(2-methoxyethyl) 5-methyl-uracil
  misc_feature (6)..(7): /note=“Phosphorothioate internucleoside linkage”
  modified_base (7)..(7): 2′-O-(2-methoxyethyl) adenine
  misc_feature (7)..(8): /note=“Phosphorothioate internucleoside linkage”
  modified_base (8)..(8): 2′-O-(2-methoxyethyl) 5-methyl-cytosine
  misc_feature (8)..(9): /note=“Phosphorothioate internucleoside linkage”
  modified_base (9)..(9): 2′-O-(2-methoxyethyl) 5-methyl-cytosine
  misc_feature (9)..(10): /note=“Phosphorothioate internucleoside linkage”
  modified_base (10)..(10): 2′-O-(2-methoxyethyl) 5-methyl-uracil
  misc_feature (10)..(11): /note=“Phosphorothioate internucleoside linkage”
  modified_base (11)..(11): 2′-O-(2-methoxyethyl) 5-methyl-uracil
  misc_feature (11)..(12): /note=“Phosphorothioate internucleoside linkage”
  modified_base (12)..(12): 2′-O-(2-methoxyethyl) adenine
  misc_feature (12)..(13): /note=“Phosphorothioate internucleoside linkage”
  modified_base (13)..(13): 2′-O-(2-methoxyethyl) adenine
  misc_feature (13)..(14): /note=“Phosphorothioate internucleoside linkage”
  modified_base (14)..(14): 2′-O-(2-methoxyethyl) 5-methyl-cytosine
  misc_feature (14)..(15): /note=“Phosphorothioate internucleoside linkage”
  modified_base (15)..(15): 2′-O-(2-methoxyethyl) 5-methyl-cytosine
  misc_feature (15)..(16): /note=“Phosphorothioate internucleoside linkage”
  modified_base (16)..(16): 2′-O-(2-methoxyethyl) 5-methyl-cytosine
  misc_feature (16)..(17): /note=“Phosphorothioate internucleoside linkage”
  modified_base (17)..(17): 2′-O-(2-methoxyethyl) adenine
  misc_feature (17)..(18): /note=“Phosphorothioate internucleoside linkage”
  modified_base (18)..(18): 2′-O-(2-methoxyethyl) guanine
gcuauuaccuuaacccag